(19) United States
(12) Patent Application Publication
(10) Pub. No.: US 2017/0137509 A1
(43) Pub. Date: May 18, 2017

(54) VACCINES AND MONOCLONAL ANTIBODIES TARGETING TRUNCATED VARIANTS OF OSTEOPONTIN AND USES THEREOF

(71) Applicant: AFFIRIS AG, Vienna (AT)

(72) Inventors: Guenther STAFFLER, Vienna (AT); Christine LANDLINGER, Vienna (AT); Marie LE BRAS, Vienna (AT)

(73) Assignee: AFFIRIS AG, Vienna (AT)

(21) Appl. No.: 15/322,356

(22) PCT Filed: Jun. 29, 2015
(86) PCT No.: PCT/EP2015/064701

§ 371 Date: Dec. 27, 2016

(30) Foreign Application Priority Data
Jun. 30, 2014 (EP) 14175005.9

(51) Int. Cl. C07K 16/24; G01N 33/68; A61K 39/00

(52) U.S. Cl. CPC C07K 16/24; A61K 39/0005; G01N 33/6863; C07K 2317/565; C07K 2317/34; C07K 2317/92; C07K 2317/24; A61K 2039/6081; C07K 2317/76; G01N 2333/52; G01N 2800/044; G01N 2800/52

(57) ABSTRACT

The present invention provides monoclonal antibodies specific for one or more truncated variants of human osteopontin and vaccines comprising at least one isolated osteopontin peptide, as well methods for manufacturing said antibodies and vaccines. Furthermore, a diagnostic method making use of said antibodies is provided. Said antibodies and vaccines are used in therapy, especially in treatment and/or prevention of Type-2 diabetes and cardiovascular disease.

The present invention relates to vaccines and monoclonal antibodies (mAbs), especially suitable for use in treatment and/or prevention of chronic inflammatory disease such as type-2 diabetes (T2D) and cardiovascular disease (CVD). Another aspect of the present invention relates to diagnostic methods using mAbs.

Sociological and economic implications of the major obesity-associated diseases.

Obesity is the epidemic challenging our society from a medical as well as an economical perspective. The escalating prevalence of obesity associated with cardiometabolic risk results in decreased life expectancy due to T2D and CVD. In the UK, for instance, rates of obese (body-mass index 30 kg/m2) have increased by 30% in women, 40% in men, and 50% in children within the last decade resulting in 23% of adults being obese in 2007 and a prognosis of 50% for 2050. According to the Centre for Disease Control (CDC) an estimated 150 million people worldwide (21 million in US alone) are affected by T2D and this number is projected to grow up to 250 million in the next two decades. At present the costs for T2D medication exceed 4% of the annual health care budget in EU countries. These costs are expected to increase dramatically over the next years. The causes underlying the obesity epidemic are still not entirely understood, but its consequences are already apparent, e.g. by the dramatic increase in T2D which nowadays even occurs in children and adolescents.

CVD is the main cause of death in the World Health Organization (WHO) European Region (consisting of 53 countries), accounting for over 4.3 million deaths each year. Although it is much harder to quantify the morbidity burden, CVD was estimated in 2006 to cost the healthcare systems of the European Union 110 billion Euros.

Current preventive and therapeutic strategies to reduce the cardiometabolic risk are inadequate and there is a need for the research community (both academic and industrial) to develop novel healthcare interventions to address this substantial biomedical challenge. Hence, there are tremendous sociological and economical implications for a treatment and prevention strategy for T2D and CVD that is relatively inexpensive and ensures patient compliance. Research has identified chronic low-grade inflammation as induced by obesity as a common mechanism that is causally involved in obesity-related insulin resistance and atherosclerosis, the basis for T2D and CVD. This offers common treatment strategies for these diseases by elimination of key factors implicated in chronic inflammation by means of immunotherapy.

Inflammation as the mechanism linking obesity, T2D, and vascular disease.

Obesity, particularly central obesity, is the prominent basis for insulin resistance that underlies features of the metabolic syndrome including dyslipidemia and hypertension. Metabolic syndrome comprises a variable combination of these factors that together add up to significant cardiometabolic risk, i.e. to develop T2D and atherosclerotic CVD. Insulin resistance is defined as impaired sensitivity to insulin of its main target organs, i.e. adipose tissue, liver, and muscle. Insulin regulates glucose uptake and circulating free fatty acid (FFA) concentrations. In adipose tissue, insulin stimulates glucose uptake and lipogenesis while inhibiting lipolysis thereby reducing FFA efflux; in liver, insulin inhibits gluconeogenesis by reducing key enzyme activities; and in skeletal muscle insulin largely enhances glucose uptake. Consequently, adipose tissue insulin resistance leads to increased circulating FFA concentrations and ectopic fat accumulation that impede insulin action in liver and skeletal muscle. Finally, gluco- and lipotoxicity induced by insulin resistance impairs insulin secretion by pancreatic β-cells that lead to overt T2D.

In the recent years, evidence has cumulated that obesity is associated with low-grade inflammation that markedly provokes the development of insulin resistance. Adipose tissue and liver are the primary source of circulating inflammatory markers in obesity that impairs insulin sensitivity. Within these organs, macrophages, possibly triggered by other cells of the immune system, drive this inflammation due to mechanisms not yet perfectly understood. The experimental findings that adipose tissue macrophages are crucially involved in the development of insulin resistance are strongly supported by recent clinical data.

A characteristic of obesity-associated conditions of insulin resistance and T2D is accelerated atherosclerosis, the underlying feature of most predominant vascular diseases. This relationship is most crucial bearing in mind that eighty percent of people with T2D will die from complications of atherosclerotic CVD resulting in an increased risk of death equivalent to 15 years of aging and also obesity per se is associated with an increased risk of myocardial infarction equivalent to over 10 years of aging.

Atherosclerosis was once identified as a lipid-storage disease, but is now recognized as an inflammatory condition of the vessel wall, characterized by infiltration of macrophages and T cells, which interact with one another and with cells of the arterial wall. Thus, pathological mechanisms of atherosclerosis recapitulate many features of the inflammatory processes observed in obesity. Additionally, a recent experimental animal study directly links obesity-induced adipose tissue inflammation to accelerated atherosclerosis indicating a common pathogenetic origin of both T2D and CVD.

Based on the common inflammatory mechanisms, simultaneous targeting of obesity-associated adipose tissue inflammation and atherosclerosis has recently been demonstrated in animal models. For instance, a small-molecule inhibitor of the adipocyte and macrophage fatty acid binding protein (aP2) effectively treated T2D and atherosclerosis, and deficiency of the inflammatory cytokine MIF has been demonstrated to reduce chronic adipose tissue inflammation and insulin resistance along with atherosclerosis in LDL receptor knockouts. Conversely, transgenic overexpression of anti-inflammatory adiponectin and cholesteryl ester hydrolase in macrophages showed similar effects. Thus, sophisticated approaches that simultaneously target major complications of obesity, i.e. insulin resistance and atherosclerosis that interrelate so closely via chronic inflammation, are viable and very attractive. (Olefsky J M, Glass C K. Annu Rev Physiol 2010; 72:219-246, PMID: 20148674; Rocha V Z, Libby P. Nat Rev Cardiol 2009; 6:399-409, PMID: 19399028)

Taken together, therefore, it is an object of the present invention to provide compounds suitable for use in prevention and therapy, especially prevention and therapy of chronic inflammatory disease such as T2D and CVD or selected conditions and/or symptoms associated with these, such as obesity-related insulin resistance and/or atherosclerosis.

It is another object of the present invention to provide methods for manufacturing said compounds and diagnostic methods using said compounds.

Consequently, an aspect of the present invention provides a monoclonal antibody (mAb) specific for one or more truncated variants of human osteopontin (Opn),

wherein the antibody is more reactive towards the one or more truncated variants than towards the full-length Opn (flOpn; SEQ ID NO: 15); and

wherein the antibody is specific for:

(A) matrix-metalloproteinase-truncated Opn (MmpOpn; SEQ ID NO: 16), wherein the antibody is more reactive towards MmpOpn than towards each of flOpn (SEQ ID NO: 15) and thrombin-truncated Opn (ThrOpn; SEQ ID NO: 17); or

(B) both MmpOpn and ThrOpn, wherein the antibody is more reactive towards each of MmpOpn and ThrOpn than towards flOpn; or

(C) ThrOpn, wherein the antibody is specific for an ThrOpn epitope with a peptide sequence selected from the group of VVYGLR, SVVYGLR and DSVVYGLR (SEQ ID NOs: 1-3), wherein, in case the antibody is specific for the epitope with the peptide sequence SVVYGLR (SEQ ID NO: 2), the antibody's variable domain of the heavy chain (V_{H}) and the antibody's variable domain of the light chain (V_{L}) comprise complementarity-determining regions (CDRs) with the following sequences:

V_{H} CDR1
(SEQ ID NO: 18)
GFSLSTYGLG,
V_{H} CDR2
(SEQ ID NO: 19)
IYWDDNK,
V_{H} CDR3
(SEQ ID NO: 20)
ARGTSPGVSFPY,
V_{L} CDR1
(SEQ ID NO: 21)
ENIYSY,
V_{L} CDR2
(SEQ ID NO: 22)
NAK,
V_{L} CDR3
(SEQ ID NO: 23)
QHHYGTPLT,

and wherein the antibody is more reactive towards ThrOpn than towards each of flOpn and MmpOpn and preferably wherein V_{H} comprises or has the sequence of SEQ ID NO: 24 and V_{L} comprises or has the sequence of SEQ ID NO: 25.

The inventive antibody is especially suitable for use in therapy, in particular for use in treatment and/or prevention of T2D, especially obesity-related insulin resistance, and/or of CVD, especially atherosclerosis, as described in the following.

Opn as a Promising Molecular Target

Opn, also named secreted phosphoprotein-1 and sialoprotein-1, is encoded by the SPP1 gene. Opn is a multifunctional protein expressed in activated macrophages and T cells, osteoclasts, hepatocytes, smooth muscle, endothelial, and epithelial cells and classified as an inflammatory cytokine. Opn functions in cell migration, particularly of monocytes/macrophages. Opn's effect on monocyte adhesion to the endothelium appears to be predominantly mediated by its action on monocytes since the major adhesion mechanisms of endothelial cells are not altered by Opn. Furthermore, Opn can induce the expression of a variety of other inflammatory cytokines and chemokines as well as matrix metalloproteases to induce matrix degradation and facilitate cell motility. These functions of Opn are based on its ability to bind adhesion molecules such as integrins and CD44, as detailed below. Beyond adhesion that is an integral component of immune cell function essential for chemotaxis and invasion of injured and inflamed tissues, integrins and CD44 transduce signals leading to immune cell migration, growth, survival, differentiation, and activation of inflammatory and adaptive immune responses.

As delineated in detail in the following paragraphs, Opn is a target molecule for treatment of obesity-associated diseases based on chronic low-grade inflammation in metabolic tissues and the vascular wall that underlie the cardiometabolic risk. Polyclonal antibodies have been successfully used in passive immunotherapy to block Opn functions and to treat inflammatory disorders including obesity-induced insulin resistance (Kiefer F W, et al. Diabetes 2010; 59:935-946; PMID: 20107108).

Yan et al. (Yan, Xiaoxiang, et al.; Cardiovasc Diabetol 9.1 (2010): 70-78.) discloses that plasma concentrations of osteopontin, but not thrombin-cleaved osteopontin, are associated with the presence and severity of nephropathy and coronary artery disease in patients with type-2 diabetes mellitus.

The Role of Opn in Obesity-Associated Insulin Resistance and Atherosclerosis

It has been demonstrated that Opn is highly upregulated upon obesity in humans and different murine models (Gomez-Ambrosi J et al. J Clin Endocrinol Metab 2007;92:3719-3727. PMID: 17595250. Kiefer F W et al. Endocrinology 2008; 149:1350-1357. PMID: 18048491). Several recent studies showed that Opn is causally involved in obesity-induced adipose tissue inflammation and insulin resistance and, in close relation, liver steatosis (for instance Kiefer F W, et al. Diabetes 2010; 59:935-946; PMID: 20107108). Strikingly, it could be demonstrated by passive immunization of mice that antibody-mediated neutralization of Opn action in vivo significantly improves insulin signaling and thus reduces insulin resistance in obesity by decreasing obesity-associated inflammation in adipose tissue and liver.

These results show macrophage activation as a mechanism of Opn action within the adipose tissue during obesity-induced adipose tissue inflammation.

Excessive abundance of Opn is not only found in obese adipose tissue. Opn is highly enriched in macrophages, smooth muscle, and endothelial cells of atherosclerotic plaques and aortic valvular lesions. Plasma Opn is a prognostic marker in patients with vascular diseases and correlates with arterial stiffness in rheumatoid arthritis patients. Knockout studies showed a direct involvement of Opn in angiotensin II-induced atherosclerosis and aneurysm formation (Bruemmer D et al. J Clin Invest 2003; 112:1318-1331. PMID: 14597759). Female mice on a mixed genetic background simultaneously deficient for apolipoprotein E (ApoE) and Opn on normal chow were significantly protected from atherosclerosis. Also female mice triple negative for ApoE, LDL receptor (LDLR), and Opn develop markedly smaller lesions and a reduced medial thickening compared to ApoE and LDLR double-knockouts. Vice versa, transgenic overexpression of Opn in high-fat high-cholesterol fed C57BL/6 mice resulted in exacerbated atherosclerotic lesions formation, increased medial thickening and neointimal formation. Moreover, Opn has also been linked to vascular disease associated with T2D by its implication in diabetic macro- and microvascular diseases and a preserved cardiac function in streptozotocin-induced diabetic mice deficient for Opn (for instance in Subramanian V, et al. Am J Physiol Heart Circ Physiol 2007; 292:H673-683. PMID: 16980342).

Role of Opn in Other Diseases

Opn is also involved the pathogenesis of systemic inflammatory or autoimmune diseases. Beyond the role in the disorders mentioned above, Opn has been shown to play a role in the pathogenesis of, amongst others, rheumatoid arthritis, cardiac fibrosis, multiple sclerosis, and to contribute to the development of experimental autoimmune encephalomyelitis. Moreover, Opn also is expressed in many malignancies such as breast and prostate cancer, osteosarcoma, glioblastoma, squamous cell carcinoma and melanoma, and plays a crucial role in promoting tumor growth and determining their metastatic potential.

Structure and Function of Opn

Opn is a highly negatively charged, extracellular matrix protein composed of about 314 amino acids (in human, 297 in mouse) and is expressed as a 33-kDa nascent protein. The predicted secondary structure of osteopontin consists of eight alpha-helices and six beta-sheet segments. Post-translational modifications leading to cell-type and condition-specific variations may account for the known variability in molecular weight.

Integrin- and CD44-Binding Functional Domains

Opn modulates a variety of cellular activities by binding and ligating integrins. Interactions between the centrally located 159RGD161 sequence and the αvβ3 integrin, which is highly expressed in macrophages and osteoclasts, have been well-documented. The according phosphorylation sites are absent from the RGD region. Contiguous with the RGD sequence, the 162SVVYGLR168 (SEQ ID NO: 2) cryptic integrin-binding site that is unmasked by thrombin cleavage is recognized by α4β1 and α9β1 integrins expressed by leukocytes. Furthermore, the metalloproteinases MMP3, MMP7, and MMP9 cleave Opn resulting in a slightly truncated form of the cryptic integrin-binding site 162SVVYG166 that again is recognized by α4β1 and α9β1 integrins expressed by leukocytes.

CD44 has been identified as a receptor for Opn through which chemotactic and cytokine responses of macrophages are controlled. However, other studies including own unpublished experiments contradict and question CD44 to be an adhesive receptor for Opn.

Proteolytic Products

Opn exhibits functionally important cleavage sites. Full length Opn can be cleaved by thrombin to expose the cryptic integrin binding sequence 162SVVYGLR168 (SEQ ID NO: 2). The mouse homologue of this cryptic domain (SLAYGLR (SEQ ID NO: 46)) is considered essential for the development of experimental arthritis (Yamamoto N, et al. J Clin Invest 2003; 112:181-188. PMID: 12865407). Data on regulation of Opn cleavage are scarce, but the thrombin-cleaved Opn has been shown to be enriched in synovial fluids of rheumatoid joints and urine of patients with rheumatoid arthritis. Sharif et al. (Sharif, Shadi A., et al. “Thrombin-activatable carboxypeptidase B cleavage of osteopontin regulates neutrophil survival and synoviocyte binding in rheumatoid arthritis.” Arthritis & Rheumatism 60.10 (2009): 2902-2912.) relates to Opn cleaved by thrombin (“OPN-R” in the document, “ThrOpn” in the present application) and OPN-R processed by thrombin-activatable carboxypeptidase B (CPB), “OPN-L”, in rheumatoid arthritis. To investigate the importance of OPN-R and OPN-L in rheumatoid arthritis, ELISAs apparently specific for these cleaved Opn forms were developed (p. 2903, col 2, §2 of the document). Briefly, rabbit polyclonal antibodies were raised against KLH-conjugated Opn peptides, among them SVVYGL (p. 2903, col 2, §2 of the document). The antibodies were epitope-mapped (p. 2904, col 1, §2 and FIG. 1 of the document). They were further purified by removal of cross-reactive antibodies by immunoadsorption (p. 2905, col 1, §1 of the document). These purified polyclonal antibodies were used to assess levels of OPN-R (ThrOpn), OPN-L and OPN in the synovial fluid of patients with osteoarthritis, psoriatic arthritis and rheumatoid arthritis (FIG. 2 of the document) and for immunostaining of synovium samples (FIG. 4 of the document). The document merely establishes anti-Opn antibodies as a research tool in rheumatoid arthritis. The document is silent on any therapeutic use of an anti-Opn antibody or an anti-Opn vaccine.

One very recent human study shows both thrombin and thrombin-cleaved Opn, as determined by Enzyme Linked Immunosorbent Assay (ELISA) from tissue extracts, to be enriched in calcified regions of stenotic aortic valves compared to non-calcified regions.

Opn is also a substrate for matrix metalloproteinases, MMP-3, MMP-7, MMP-2, and MMP-9. Notably, proteinase activities of eleven MMPs have been implicated in atherogenesis, and it has been demonstrated increased expression of several MMPs including MMP-7 and -9 in murine and human obesity (Unal R, et al. J Clin Endocrinol Metab 2010; 95:2993-3001. PMID: 20392866). Many of these MMPs are secreted by or expressed on the surface of macrophages and MMP-9 activity may even be induced by Opn. Of note, the 166GL167 cleavage site of MMP-9, -7 and -3 is located in closest proximity to the 168RS169 thrombin cleavage site. Hence, MMP cleavage unmasks the cryptic integrin binding site similar to thrombin. This is shown by a study showing that MMP-3 or MMP-7 cleavage of Opn potentiated the function of Opn as an adhesive and migratory stimulus for murine tumor cells and macrophages, respectively. Moreover, by analysis of effects of recombinant peptides, the 162SVVYG166 region was suggested to be sufficient to mediate binding to β1 integrins.

Epitopes Targeted to Neutralize Opn Function

In addition to successful application of polyclonal anti-Opn antibodies in treatment of obesity-associated inflammation and insulin resistance and, for instance, glomerular fibrosis, several mAbs have been used in passive immunization studies to successfully block Opn function:

anti-SLAYGLR (SEQ ID NO: 46) (the mouse homologue to human 162SVVYGLR168—SEQ ID NO: 2) antibody M5 abrogated monocyte migration and inhibited the proliferation of synovium, bone erosion, and inflammatory cell infiltration in arthritic joints in mice (Yamamoto N, et al. J Clin Invest 2003; 112:181-188. PMID: 12865407). A chimeric anti-SVVYGLR mAb (C2K1, SEQ ID NO: 2) was successfully tested in a non-human primate rheumatoid arthritis model (Yamamoto N, et al. Int Immunopharmacol 2007; 7:1460-1470. PMID: 17761350).

mAb53 inhibited RGD-dependent cellular adhesion to Opn via an epitope only present before thrombin cleavage (Bautista D S, et al. J Biol Chem 1994; 269:23280-23285. PMID: 8083234). Another MAb (Opn 1.2) inhibited RGD-function by binding to Asp113-Arg128 and thus inducing intramolecular changes (Yamaguchi Y, Hanashima S, Yagi H, et al. NMR characterization of intramolecular interaction of osteopontin, an intrinsically disordered protein with cryptic integrin-binding motifs. Biochem Biophys Res Commun 2010; 393:487-491. PMID: 20152802).

In vivo application of these antibodies has never been disclosed.

A humanized mAb against 212NAPSD216 has been demonstrated to be effective in inhibiting the cell adhesion, migration, invasion and colony formation of a human breast cancer cell line and to significantly suppress primary tumor growth and spontaneous metastasis in a mouse lung metastasis model of human breast cancer (Dai J, et al. Cancer Immunol Immunother 2010; 59:355-366. PMID: 19690854).

The mAb 23C3 specific for the N-terminal 41ATWLNPDPSQKQ52 motif of Opn reduced the production of inflammatory cytokines and promoted apoptosis of activated T cells in collagen-induced arthritis (Fan K, et al. Arthritis Rheum 2008; 58:2041-2052. PMID: 18576331). 31QLYNKYP37 is another N-terminal sequence that was targeted by a mAb (F8E11), which blocked Opn induced T cell activation and migration in vitro (Dai J, et al. Biochem Biophys Res Commun 2009; 380:715-720. PMID: 19285028).

In summary, from these findings it is concluded that passive immunization by mAb against small but functionally relevant Opn epitopes are effective to interfere with chronic inflammatory reactions.

In addition, mAbs against Opn are known from the following document:

Kon et al. (Kon, Shigeyuki, et al. “Mapping of functional epitopes of osteopontin by monoclonal antibodies raised against defined internal sequences.” Journal of Cellular Biochemistry 84.2 (2002): 420-432.) discloses five mAbs from hybridomas of splenocytes obtained from mice immunised with various Opn peptides (see FIG. 1 of the document) coupled to thyroglobulin (p. 423, col 1). The mAbs were used to analyse urine samples from healthy men (FIG. 3 of the document) and supernatant from tumor cell lines (FIG. 4 of the document) for the presence of selected Opn epitopes. The document merely establishes anti-Opn mAbs as a research tool in Opn-related physiological and pathological processes (see also the last three sentences of the discussion section in the document). Not even a single therapeutic use of an anti-Opn mAb (or an anti-Opn vaccine) is suggested.

Safety Considerations for the Intended Vaccination Against Opn

Phenotype of Opn (Spp1) Knockouts

Safety is an important issue in patients that are at considerable cardiometabolic risk but are still not acutely ill. Notably, two independent mouse strains homozygous for the targeted Spp1 deletion are viable, fertile, normal in size and do not display any gross physical or behavioral abnormalities (Liaw L, et al. J Clin Invest 1998; 101:1468-1478. PMID: 9525990. Rittling S R et al. Journal of Bone and Mineral Research 1998; 13:1101-1111. PMID: 9661074).

Conclusions on Opn as a Target for Immunotherapy

T2D and CVD share a common inflammatory basis and Opn is a key molecule that has shown functional implications in insulin resistance and atherogenesis in vitro and in vivo. Hence, Opn is a target for immunotherapy with reasonable considerations on efficacy and safety.

Vaccination strategies represent one of the most effective medical interventions in human history. While during the last century the development of prophylactic vaccination against infectious diseases dominated this area, in recent years more and more attention has been put in developing therapeutic vaccines, both passive and active. A number of antibodies has recently been introduced in clinical medicine to target key molecules in inflammatory and neoplastic diseases providing extraordinary specificity and efficacy. Beside such a passive immunotherapeutic approach, active vaccination approaches appear to be effective since in numerous animal studies and first clinical trials therapeutic B-cell vaccines are shown to hold potential as affordable and effective therapeutic option for treating a variety of non-infectious human diseases. The concept of active therapeutic vaccination represents, therefore, a strategy of immunotherapy that is considered to be applied to almost all diseases where a passive immunotherapeutic approach has been successful. The principle is to design a vaccine, which can trigger an immune response against an endogenous protein that is pathogenic and over-expressed in a given disease. Most of the currently available therapeutic vaccines use either the self-protein or a self-epitope derived from such a protein, or a modified form of the epitope (often referred to as mimotope) coupled to a carrier, both engineered to induce strong humoral immune responses to the target protein to neutralize its pathogenic effect.

Several alternative Opn epitopes including receptor binding sites, putative neoepitopes induced by proteolytic cleavage, and epitopes whose binding induce functional conformation changes are target sequences for a successful immunotherapeutic strategy against Opn.

In the course of the present invention, many Opn epitopes (among them all epitopes listed in Table 1) were evaluated for their immunogenicity, as well as the selectivity of sera/antibodies raised thereon for specific truncated variants of Opn and the function of said sera/antibodies (FIGS. 1-5). It was found that epitopes exist that can be used to raise a mAb specific for one or more truncated variants of human osteopontin (Opn) where, surprisingly, the antibody is substantially more reactive (and specific) towards the one or more truncated variants than towards the full-length Opn (flOpn). (See for instance FIG. 3)

Therefore, an aspect of the present invention provides a mAb specific for one or more truncated variants of human osteopontin (Opn), which mAb is more reactive towards the one or more truncated variants than towards the full-length Opn (flOpn).

This antibody is specific for:

(A) matrix-metalloproteinase-truncated Opn (MmpOpn; SEQ ID NO: 16), wherein the antibody is more reactive towards MmpOpn than towards each of flOpn (SEQ ID NO: 15) and thrombin-truncated Opn (ThrOpn; SEQ ID NO: 17); or

(B) both MmpOpn and ThrOpn, wherein the antibody is more reactive towards each of MmpOpn and ThrOpn than towards flOpn; or

(C) ThrOpn, wherein the antibody is specific for an ThrOpn epitope with a peptide sequence selected from the group of VVYGLR, SVVYGLR and DSVVYGLR (SEQ ID NOs: 1-3), wherein, in case the antibody is specific for the epitope with the peptide sequence SVVYGLR, the antibody's variable domain of the heavy chain (V_{H}) and the antibody's variable domain of the light chain (V_{L}) comprise complementarity-determining regions (CDRs) with the following sequences (i.e. the CDRs of mAb 4-4-2 generated in the course of this invention, cf. Table 2):

V_{H} CDR1
(SEQ ID NO: 18)
GFSLSTYGLG,
V_{H} CDR2
(SEQ ID NO: 19)
IYWDDNK,
V_{H} CDR3
(SEQ ID NO: 20)
ARGTSPGVSFPY,
V_{L} CDR1
(SEQ ID NO: 21)
ENIYSY,
V_{L} CDR2
(SEQ ID NO: 22)
NAK,
V_{L} CDR3
(SEQ ID NO: 23)
QHHYGTPLT,

and wherein the antibody is more reactive towards ThrOpn than towards each of flOpn and MmpOpn and preferably wherein V_{H} comprises or has the sequence of SEQ ID NO: 24 and V_{L} comprises or has the sequence of SEQ ID NO: 25. The antibody's preferred isotype is immunoglobulin G (IgG) and the preferred light chain type is kappa.

CDRs represent variable regions of antibodies, with which the antibody binds to its specific epitope. The type and number of heavy chain determines the class of antibody, i.e. IgA, IgD, IgE, IgG and IgM antibodies, respectively. Antibodies contain also two identical light chains, which can be of lambda or kappa type.

Being specific for truncated variants of Opn (MmpOpn, ThrOpn, or both) while showing less reactivity towards the full-length protein (flOpn) allows for a very targeted approach in therapy. This will reduce side-effects in a patient undergoing treatment with the antibody of the present invention. The antibodies of the present invention are carefully selected to show such a beneficial specificity (see also Examples).

See Table 1 for epitopes studied in the course of the present invention.

An antibody specific for a peptide comprising SVVYGLR (SEQ ID NO: 2) is for instance known from the WO 2009/023411 A1, US 2007/0274993 A1, US 2006/0002923 A1, and US 2004/0234524 A1. However, all of these documents are silent in respect to the inventive feature of providing a mAb specific for SVVYGLR (SEQ ID NO: 2), wherein the mAb is more reactive towards ThrOpn than towards each of flOpn and MmpOpn. Furthermore, all of these documents are completely silent in respect to an antibody specific for MmpOpn or for both MmpOpn and ThrOpn.

Other monoclonal anti-Opn antibodies are also known, however, they do not possess the beneficial properties of the inventive antibody described herein (in particular strong reactivity to MmpOpn or ThrOpn or both, while being less reactive to flOpn):

2K1, C2K1 (Yamamoto N, et al. Int Immunopharmacol 2007; 7:1460-1470. PMID: 17761350): The murine mAb clone 2K1 was generated against the human Opn peptide VDTYDGRGDSVVYGLRS (SEQ ID NO: 48). In vitro, the clone 2K1 was shown to inhibit the RGD-dependent cell adhesion to full length Opn as well as the RGD-independent cell adhesion to a recombinant N-terminal Opn (equivalent to ThrOpn, aa1-168). Furthermore it inhibits the α9-mediated cell migration towards full-length, thrombin-cleaved and the recombinant N-terminal Opn. The clone 2K1 is therefore able to recognize the cryptic epitope of human Opn, SVVYGLR (SEQ ID NO: 2). The later developed mAb C2K1 is a chimeric antibody, in which the variable region of 2K1 was fused with human IgG1 constant region. In vitro, C2K1 was shown to inhibit human and monkey monocyte migration towards human and monkey N-terminal Opn respectively. In a therapeutic in vivo approach, C2K1 could ameliorate established collagen-induced arthritis in non-human primates. 2K1 and C2K1 recognize the cryptic epitope of Opn and are functional, but they do not differentially distinguish between flOpn and ThrOpn. Their reactivity and functionality against MmpOpn is not disclosed.

mAb53, mAb87-B (Bautista D S, et al. J Biol Chem 1994; 269:23280-23285. PMID: 8083234): The mAbs mAb53 and mAb87-B were raised against a bacterially produced recombinant GST-human Opn (GST-hOpn). As they were raised against a full-length protein, they do not possess the beneficial properties of the inventive antibody.

34E3: The murine mAb clone 34E3 was generated against the human Opn peptide CSVVYGLR (SEQ ID NO: 49) and specifically recognizes the C-terminal amino acid sequence YGLR (SEQ ID NO: 50). 34E3 cross-reacts with Thrombin-cleaved murine Opn, rabbit Opn and human Opn but not with the uncleaned Opn. 34E3 is able to inhibit adhesion of the murine melanoma cell line B16.F10 towards murine Opn peptides VDVPNGRGDSLAYGLR (SEQ ID NO: 47) and SLAYGLR (SEQ ID NO: 46) but not towards GRGDS indicating an adhesion inhibition specific for α4- and α9-integrins. Functional data for human Opn sequences were not shown. Neither were reactive and functional against MmpOpn (see US 2011/312000 A1).

In a further preferable embodiment of the present invention, the antibody is specific for an MmpOpn epitope with a peptide sequence selected from the group of GDSVVYG, RGDSVVYG and DGRGDSVVYG (SEQ ID NOs: 7-9), or for a MmpOpn/ThrOpn epitope with a peptide sequence selected from the group of TYDGRGDSVVYG (SEQ ID NO: 10) and PTVDTYDGRGDS (SEQ ID NO: 14).

The sera generated from the epitopes most suitable for the present invention were further scrutinised for their biological function (FIG. 2). The three epitopes that were exceptionally suitable were used to generate selective mAbs (characterised in FIG. 3-5; see also Table 2 and Examples). Therefore, further embodiments of the present invention disclose exceptionally suitable antibodies, in particular their CDR sequences or their V_{H} or V_{L} sequence (the inventive sequences of an antibody specific for SVVYGLR (SEQ ID NO: 2) are disclosed above.):

One of these preferable embodiments is the antibody specific for the epitope with the peptide sequence GDSVVYG and the CDRs of the antibody comprise the following sequences (i.e. the CDRs of mAb 7-5-4 and mAb 9-3-1 generated in the course of this invention, cf. Table 2; mAb 7-5-4 and mAb 9-3-1 turned out to be identical):

V_{H} CDR1
(SEQ ID NO: 26)
GITFNTNG,
V_{H} CDR2
(SEQ ID NO: 27)
VRSKDYNFAT,
V_{H} CDR3
(SEQ ID NO: 28)
VRPDYYGSSFAY,
V_{L} CDR1
(SEQ ID NO: 29)
QSIVHSNGNTY,
V_{L} CDR2
(SEQ ID NO: 30)
KVS,
V_{L} CDR3
(SEQ ID NO: 31)
FQGSHVPWT,

and preferably wherein V_{H} comprises or has the sequence of SEQ ID NO: 32 and V_{L} comprises or has the sequence of SEQ ID NO: 33. The antibody's preferred isotype is immunoglobulin G (IgG) and the preferred light chain type is kappa.

Another of these preferable embodiments is the antibody specific for the epitope with the peptide sequence TYDGRGDSVVYG and the CDRs of the antibody comprise the following sequences (i.e. the CDRs of mAb 21-5-4 generated in the course of this invention, cf. Table 2):

V_{H} CDR1
(SEQ ID NO: 34)
GFSLSTSGLG,
V_{H} CDR2
(SEQ ID NO: 35)
ISWDDSK,
V_{H} CDR3
(SEQ ID NO: 363)
ARSGGGDSD,
V_{L} CDR1
(SEQ ID NO: 37)
SSVNS,
V_{L} CDR2
(SEQ ID NO: 38)
DTS,
V_{L} CDR3
(SEQ ID NO: 39)
FQGSGYPLT

and preferably wherein V_{H} comprises or has the sequence of SEQ ID NO: 40 and V_{L} comprises or has the sequence of SEQ ID NO: 41. The antibody's preferred isotype is immunoglobulin G (IgG) and the preferred light chain type is kappa.

Although a mutation in CDRs may lead to a decrease in affinity or selectivity, a limited amount of mutations can be tolerable or even beneficial in terms of affinity or selectivity. Therefore, in another preferable embodiment of the present invention, in the inventive antibody, three, preferably two, more preferably one, of the amino-acids of the CDR or V_{H} or V_{L} are mutated into any other amino-acid.

It is especially beneficial if the inventive antibody has a low cross-reactivity, therefore another preferred embodiment of the present invention provides the inventive antibody wherein
in case of the antibody being specific for MmpOpn, the antibody is more than N times more reactive towards MmpOpn than towards each of flOpn and ThrOpn; and
in case of the antibody being specific for both MmpOpn and ThrOpn, the antibody is more than N times more reactive towards each of MmpOpn and ThrOpn than towards flOpn; and
in case of the antibody being specific for ThrOpn, the antibody is more than N times more reactive towards ThrOpn than towards each of flOpn and MmpOpn; and

wherein N is more than 1.5, preferably more than 2, more preferably more than 3, even more preferably more than 5, most preferably more than 10.

ELISA assays for testing the (cross-)reactivity of antibodies (or sera) are widely used in the art. Thus, preferably, the reactivity of the antibody towards MmpOpn, ThrOpn and flOpn, respectively, is measured by ELISA on a plate coated by MmpOpn, ThrOpn and flOpn, respectively, after blocking with 1% BSA comprising the following conditions:

concentration of the antibody: 0.25 μg/ml,

concentration of the secondary, HRP-coupled antibody: 0.1 μg/ml,

HRP substrate: ABTS and 0.1% hydrogen peroxide,

read-out: absorbance at 405 nm.

Antibodies can be classified by their dissociation constant K_{d} in regard to the respective epitope (or ligand) (which is also called “affinity”) and by their off-rate value in regard to the respective epitope (or ligand). Both terms (and how they are measured) are well-known in the art. If not taking other parameters into account, the higher the affinity (i.e. the lower the K_{d} is) and/or the lower the off-rate value of the antibody is, the more suitable it is.

Hence, in another preferable embodiment of the invention, the dissociation constant K_{d} of the antibody in regard to the respective epitope and/or in regard to the respective Opn protein is lower than 50 nM, preferably lower than 20 nM, more preferably lower than 10 nM, even more preferably lower than 5 nM, most preferably lower than 2 nM. Moreover, in another preferable embodiment of the invention, the off-rate value of the antibody in regard to the respective epitope and/or in regard to the respective Opn protein is lower than 5×10⁻³s⁻¹, preferably lower than 3×10⁻³s⁻¹, more preferably lower than 1×10⁻³s⁻¹, even more preferably lower than 1×10⁻⁴s⁻¹. Table 3 lists determined dissociation and off-rate values of selected inventive antibodies.

The antibody of the present invention is preferably humanised. Methods to obtain such antibodies are well known in the art. One method is to insert the variable regions disclosed herein into a human antibody scaffold (see e.g. Hou S, et al., J Biochem 2008, PMID: 18424812).

A “humanized” antibody refers to a chimeric antibody comprising amino acid residues from non-human HVRs and amino acid residues from human Frs (fixed regions). “Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. In one preferred embodiment, a murine HVR is grafted into the framework region of a human antibody to prepare the “humanized antibody”. The murine variable region amino acid sequence is aligned to a collection of human germline antibody V-genes, and sorted according to sequence identity and homology. The acceptor sequence is selected based on high overall sequence homology and optionally also the presence of the right canonical residues already in the acceptor sequence. The germline V-gene encodes only the region up to the beginning of HVR3 for the heavy chain, and till the middle of HVR3 of the light chain. Therefore, the genes of the germline V-genes are not aligned over the whole V-domain. The humanized construct comprises the human frameworks to 3, the murine HVRs, and the human framework 4 sequence derived from the human JK4, and the JH4 sequences for light and heavy chain, respectively. Before selecting one particular acceptor sequence, the so-called canonical loop structures of the donor antibody can be determined. These canonical loop structures are determined by the type of residues present at the so-called canonical positions. These positions lie (partially) outside of the HVR regions, and should be kept functionally equivalent in the final construct in order to retain the HVR conformation of the parental (donor) antibody. In WO 2004/006955 A1 a method for humanizing antibodies is reported that comprises the steps of identifying the canonical HVR structure types of the HVRs in a non-human mature antibody; obtaining a library of peptide sequence for human antibody variable regions; determining the canonical HVR structure types of the variable regions in the library; and selecting the human sequences in which the canonical HVR structure is the same as the non-human antibody canonical HVR structure type at corresponding locations within the non-human and human variable regions. Summarizing, the potential acceptor sequence is selected based on high overall homology and optionally in addition the presence of the right canonical residues already in the acceptor sequence. In some cases simple HVR grafting only result in partial retention of the binding specificity of the non-human antibody. It has been found that at least some specific non-human framework residues are required for reconstituting the binding specificity and have also to be grafted into the human framework, i.e. so called “back mutations” have to be made in addition to the introduction of the non-human HVRs (see e.g. Queen et al., PNAS 86 (1989), 10029-10033). These specific framework amino acid residues participate in FR-HVR interactions and stabilized the conformation (loop) of the HVRs. In some cases also forward-mutations are introduced in order to adopt more closely the human germline sequence. Thus “humanized antibody of to the invention” (which is e.g. of mouse origin) refers to an antibody, which is based on the mouse antibody sequences in which the VH and VL are humanized by above described standard techniques (including HVR grafting and optionally subsequent mutagenesis of certain amino acids in the framework region and the HVR-H1, HVR-H2, HVR-L1 or HVR-L2, whereas HVR-H3 and HVR-L3 remain unmodified).

In certain embodiments, an antibody provided herein is a chimeric antibody. Certain chimeric antibodies are described, e.g., in U.S. Pat. No. 4,816,567 A; and Morrison et al., Proc. Natl. Acad. Sci. USA 81 (1984) 6851-6855). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, an antibody provided herein is a human antibody. Human antibodies can be produced using various techniques known in the art. Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23 (2005) 1117-1125, U.S. Pat. Nos. 6,075,181 and 6,150,584 describing XENOMOUSE™ technology; U.S. Pat. No. 5,770,429 A describing HuMab® technology; U.S. Pat. No. 7,041,870 A describing K-M MOUSE® technology, and US 2007/0061900 A1, describing VelociMouse® technology. Human variable regions from intact antibodies generated by such animals may be further modified, e.g., by combining with a different human constant region.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat, E. A. et al, Sequences of Proteins of Immunological Interest, 5th ed., Bethesda Md. (1991), NIH Publication 91-3242, Vols. 1-3. In one embodiment, for the VL, the subgroup is subgroup kappa I as in Kabat et al, supra. In one embodiment, for the VH, the subgroup is subgroup III as in Kabat et al, supra.

Human antibodies can also be made by hybridoma-based methods. Human myeloma and mouse-human heteromyeloma cell lines for the production of human monoclonal antibodies have been described. Human antibodies generated via human B-cell hybridoma technology are also described in Li et al, PNAS 103 (2006) 3557-3562. Additional methods include those described, for example, in U.S. Pat. No. 7,189,826 (describing production of monoclonal human IgM antibodies from hybridoma cell lines) or made by the Trioma technology. Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Antibodies according to the present invention may also be isolated by screening combinatorial libraries for antibodies with the desired activity or activities. For example, a variety of methods are known in the art for generating phage display libraries and screening such libraries for antibodies possessing the desired binding characteristics. Such methods are further described, e.g., in Fellouse, PNAS (2004) 12467-12472.

In certain phage display methods, repertoires of VH and VL genes are separately cloned by polymerase chain reaction (PCR) and recombined randomly in phage libraries, which can then be screened for antigen-binding phage. Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization. Finally, naive libraries can also be made synthetically by cloning non-rearranged V-gene segments from stem cells, and using PCR primers containing random sequence to encode the highly variable CDR3 regions and to accomplish rearrangement in vitro. Further publications describing human antibody phage libraries include, for example: U.S. Pat. No. 5,750,373 A, US 2005/0079574 A1, US 2005/0119455 A1, US 2005/0266000 A1, US 2007/0117126 A1, US 2007/0160598 A1, US 2007/0237764 A1, US 2007/0292936 A1, and US 2009/0002360 A1. Antibodies or antibody fragments isolated from human antibody libraries are considered human antibodies or human antibody fragments herein.

In certain embodiments, an antibody provided herein is a multi-specific antibody, e.g. a bi-specific antibody. Multi-specific antibodies are monoclonal antibodies that have binding specificities for at least two different sites. In certain embodiments, one of the binding specificities is for an epitope mentioned herein and the other is for another epitope mentioned herein or any other antigen. Bi-specific antibodies can be prepared as full length antibodies or antibody fragments. Techniques for making multi-specific antibodies include, but are not limited to, recombinant co-expression of two immunoglobulin heavy chain-light chain pairs having different specificities (WO 93/08829 A, U.S. Pat. No. 5,731,168 A). Multi-specific antibodies may also be made by engineering electrostatic steering effects for making antibody Fc-heterodimeric molecules (WO 2009/089004 A); cross-linking two or more antibodies or fragments (e.g. U.S. Pat. No. 4,676,980 A); using leucine zippers to produce bi-specific antibodies; using “diabody” technology for making bi-specific antibody fragments (e.g., Holliger et al, PNAS 90 (1993) 6444-6448); and using single-chain Fv (sFv) dimers; and preparing tri-specific antibodies. Engineered antibodies with three or more functional antigen binding sites, including “Octopus antibodies” are also included herein (e.g. US 2006/0025576 A1). The antibody or fragment herein also includes a “Dual Acting Fab” or “DAF” comprising an antigen binding site that binds to an epitope mentioned herein as well as another, different antigen (see US 2008/0069820 A1, for example). The antibody or fragment herein also includes multi-specific antibodies described in WO 2009/080251 A, WO 2009/080252 A, WO 2009/080253 A, WO 2009/080254 A, WO 2010/112193 A, WO 2010/115589 A, WO 2010/136172 A, WO 2010/145792 A, and WO 2010/145793 A.

Functional fragments of antibodies are fragments that are also able to bind the respective epitope. They offer advantages over the full-size antibody due to considerations such as chemical stability, pharmaceutical half-life, dosing or simpler production. Such fragments are for instance Fab fragments, single-domain antibodies and binding domains derived from the constant region of an antibody (such as an Fcab™)

Thus, another preferred embodiment of the present invention is an antibody fragment, preferably a single-domain antibody, wherein the fragment is specific for:

(A) MmpOpn (SEQ ID NO: 16), wherein the fragment is more reactive towards MmpOpn than towards each of flOpn (SEQ ID NO: 15) and ThrOpn (SEQ ID NO: 17); or

(B) both MmpOpn and ThrOpn, wherein the fragment is more reactive towards each of MmpOpn and ThrOpn than towards flOpn; or

(C) ThrOpn, wherein the fragment is more reactive towards ThrOpn than towards each of flOpn and MmpOpn.

The inventive fragments also comprise: “Kappa bodies” (III et al., Protein Eng. 10: 949-57 (1997)), “Minibodies” (Martin et al., EMBO J. 13: 5303-9 (1994)), “Diabodies” (Holliger et al., Proc. Natl. Acad. Sci. USA 90: 6444-6448 (1993)), or “Janusins” (Traunecker et al., EMBO J. 10:3655-3659 (1991) and Traunecker et al., Int. J. Cancer (Suppl.) 7:51-52 (1992)). They may be prepared using standard molecular biological techniques following the teachings of the specification.

An overview over such engineered antibody fragments that are suitable in the present invention is also given in Hollinger & Hudson (Nat Biotechnol. 2005, PMID: 16151406).

In another preferred embodiment of the present invention, at least one amino-acid residue, an N-terminus and/or a C-terminus of the antibody (or antibody fragment) of the present invention is chemically modified according to methods known in the art. Such modifications comprise one or more of glycosylation, pegylation, biotinylation, alkylation, hydroxylation, adenylation, phosphorylation, succinylation, oxidation, or acylation, in particular acetylation. This improves the pharmaceutical properties (such as solubility, half-life, activity) of the antibody of the present invention.

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In certain embodiments, antibody variants having one or more amino acid substitutions are provided. Sites of interest for substitutional mutagenesis include the HVRs and FRs. Conservative substitutions are shown in Table 1 under the heading of “preferred substitutions”. More substantial changes are provided below in reference to amino acid side chain classes. Amino acid substitutions may be introduced into an antibody of interest and the products screened for a desired activity, e.g., retained/improved antigen binding, decreased immunogenicity, or improved ADCC or CDC.

| Original | Exemplary | Preferred |
|---|---|---|
| Residue | Substitution | Substitution |
| Ala (A) | Val, Leu, lle, | Val |
| Arg (R) | Lys, Gln, Asn | Lys |
| Asn (N) | Gln, His, Asp, Lys, Arg | Gln |
| Asp (D) | Glu, Asn | Glu |
| Cys (C) | Ser, Ala | Ser |
| Gln (Q) | Asn, Glu | Asn |
| Glu (E) | Asp, Gln | Asp |
| Gly (G) | Ala | Ala |
| His (H) | Asn, Gln, Lys, Arg | Arg |
| Ile (I) | Leu, Val, Met, Ala, Phe, Norleucine | Leu |
| Leu (L) | Norleucine, Ile, Val, Met, Ala, Phe | lle |
| Lys (K) | Arg, Gln, Asn | Arg |
| Met (M) | Leu, Phe, Ile | Leu |
| Phe (F) | Trp, Leu, Val, Ile, Ala, Tyr | Tyr |
| Pro (P) | Ala | Ala |
| Ser (S) | Thr | Thr |
| Thr (T) | Val, Ser | Ser |
| Trp (W) | Tyr, Phe | Tyr |
| Tyr (Y) | Trp, Phe, Thr, Ser | Phe |
| Val (V) | Ile, Leu, Met, Phe, Ala, Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gin;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class. One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity). Alterations (e.g., substitutions) may be made in HVRs, e.g., to improve antibody affinity. Such alterations may be made in HVR “hotspots,” i.e., residues encoded by codons that undergo mutation at high frequency during the somatic maturation process, and/or SDRs (a-CDRs), with the resulting variant VH or VL being tested for binding affinity. Affinity maturation is performed by constructing and reselecting from secondary libraries. In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modelling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g. conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions. A useful method for identification of residues or regions of an antibody that may be targeted for mutagenesis is called “alanine scanning mutagenesis”. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighbouring residues may be targeted or eliminated as candidates for substitution. Variants may be screened to determine whether they contain the desired properties. Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

It is advantageous to provide the antibody of the present invention in a pharmaceutical composition, for instance to increase the stability of the antibody in storage. Consequently, another preferred embodiment provides a pharmaceutical composition comprising the antibody or fragment of the invention, further comprising one or more excipients.

The term “pharmaceutical composition” as refers to any composition or preparation that contains an antibody, as defined above, which ameliorates, cures or prevents the conditions described herein. In particular, the expression “a pharmaceutical composition” refers to a composition comprising an antibody according to the present invention and a pharmaceutically acceptable excipient. Suitable excipients are known to the person skilled in the art, for example water (especially water for injection), saline, Ringer's solution, dextrose solution, buffers, Hank solution, vesicle forming compounds (e.g. lipids), fixed oils, ethyl oleate, 5% dextrose in saline, substances that enhance isotonicity and chemical stability, buffers and preservatives. Other suitable excipients include any compound that does not itself induce the production of antibodies in the patient that are harmful for the patient. Examples are well tolerable proteins, polysaccharides, polylactic acids, polyglycolic acid, polymeric amino acids and amino acid copolymers. This pharmaceutical composition or the antibody of the present invention can (as a drug) be administered via appropriate procedures known to the skilled person to a patient in need thereof (i.e. a patient having or having the risk of developing the diseases or conditions mentioned herein). The patient is preferably human.

The preferred route of administration of the inventive composition is parenteral administration, in particular through intravenous or subcutaneous administration. For parenteral administration, the pharmaceutical composition of the present invention is provided in injectable dosage unit form, eg as a solution, suspension or emulsion, formulated in conjunction with the above-defined pharmaceutically acceptable excipients. The dosage and method of administration, however, depends on the individual patient to be treated.

The antibodies according to the present invention can be administered in any suitable dosage known from other mAB dosage regimen or specifically evaluated and optimised for a given individual. For example, the mABs according to the present invention may be provided as dosage form (or applies as dosage of) in an amount from 1 mg to 10 g, preferably 50 mg to 2 g, in particular 100 mg to 1 g. Usual dosages can also be determined on the basis of kg body weight of the patient, for example preferred dosages are in the range of 0.1 mg to 100 mg/kg body weight, especially 1 to 10 mg/body weight (per administration session).

As the preferred mode of administration of the inventive composition is parenteral administration, the pharmaceutical composition according to the present invention is preferably liquid or ready to be dissolved in liquid such sterile, de-ionised or distilled water or sterile isotonic phosphate-buffered saline (PBS). Preferably, 1000 μg (dry-weight) of such a composition comprises or consists of 0.1-990 μg, preferably 1-900 μg, more preferably 10-200 μg antibody according to the present invention, and optionally 1-500 μg, preferably 1-100 μg, more preferably 5-15 μg (buffer) salts (preferably to yield an isotonic buffer in the final volume), and optionally 0.1-999.9 μg, preferably 100-999.9 μg, more preferably 200-999 μg other excipients. Preferably, 100 mg of such a dry composition is dissolved in sterile, de-ionised/distilled water or sterile isotonic phosphate-buffered saline (PBS) to yield a final volume of 0.1-100 ml, preferably 0.5-20 ml, more preferably 1-10 ml.

As some of the epitopes found and/or characterised in the course of the present invention proved exceptionally suitable for antibody generation (in terms of selectivity and immunogenicity), another aspect of the invention concerns using the same epitopes in an active immunisation approach (i.e. vaccination).

Consequently, this aspect of the present invention provides a vaccine, comprising at least one isolated Opn peptide:

(A) with one or more sequences selected from the group of GDSVVYG, RGDSVVYG and DGRGDSVVYG (SEQ ID NOs: 7-9) and GRGDSVVYG (SEQ ID NO: 55); and/or

(B) with one or more sequences selected from the group of TYDGRGDSVVYG (SEQ ID NO: 10), VDTYDGRGDSVV (SEQ ID NO: 13), PTVDTYDGRGDS (SEQ ID NO: 14) and DTYDGRGDSVVY (SEQ ID NO: 56) and TVDTYDGRGDSV (SEQ ID NO: 57), wherein the peptide, especially the peptide with the sequence VDTYDGRGDSVV (SEQ ID NO: 13) or PTVDTYDGRGDS (SEQ ID NO: 14), is preferably amidated at its C-terminus (resulting in R—COO—NH₂, wherein R is the peptide without its C-terminal carboxyl group) and/or

(C) with one or more sequences selected from the group of VVYGLR, SVVYGLR and DSVVYGLR (SEQ ID NOs: 1-3) and GDSVVYGLR (SEQ ID NO: 58);

and wherein the peptides are coupled to a pharmaceutically acceptable carrier. These epitopes are highly suitable because they can be expected to elicit a strong and selective response against ThrOpn, MmpOpn, or both, in the patient, while eliciting a lower response towards flnOpn.

Prior to the present invention, no vaccination approach targeting specifically the cryptic domain of Opn made accessible either by Thr- or MMP-cleavage, or both, or addressing the RGD region in the context of the cryptic domain was known to one of ordinary skill in the art.

The WO 02/25285 A1 relates to a prognostic indicator for metastasis comprising an antibody directed against Opn. The document teaches on p. 6ff that a vaccine comprising an antigenic peptide will generate an antibody directed against Opn. The document merely relates to the treatment of cancer. Moreover, the document only explicitly mentions an N-terminal Opn sequence as a sequence where the peptide can be derived from, which is more than 100 amino-acid residues away from the peptides of present inventive vaccine. In particular, the document teaches the peptide is preferably derived from N-terminus, “since the amino terminus is extracellularly exposed”—seemingly oblivious of the fact that Opn is secreted and optionally processed by proteases such as thrombin.

Furthermore, immune sera induced by vaccines of the present invention are shown herein to be specific for truncated variants of Opn (MmpOpn, ThrOpn, or both) while showing less reactivity towards the full-length protein (flOpn) (cf. for instance FIG. 1 and Example 1). This allows for a very targeted approach in therapy, which will reduce side-effects in a patient undergoing treatment with the vaccine of the present invention.

The “vaccine” composition according to the present invention may therefore also be regarded as an “immunogenic composition”, i.e. a composition that is eliciting an immune response in a human individual to whom the composition is applied. It is, however, known that the power to elicit immune response in a human individual may vary significantly within a population, for the purpose of the present invention it is therefore referred to an “immunogenic composition” if in at least 10%, preferably in at least 20%, more preferred in at least 30%, especially at least 50%, of the individuals of a given population, an immune reaction after delivery of the immunogenic compositions according to the present invention is detectable, e.g. antibodies specific for the peptides delivered are formed by the individual's immune system.

Preferably, one or more peptides of the vaccine of the present invention, especially the peptides with the sequences TYDGRGDSVVYG and PTVDTYDGRGDS, are amidated at their C-termini, in order to direct the antibody response to a more central part of the peptide used for immunization. In another preferred embodiment, one or more peptides of the vaccine of the present invention are otherwise modified as known in the art. Such modifications comprise one or more of glycosylation, pegylation, biotinylation, alkylation, hydroxylation, adenylation, phosphorylation, succinylation, oxidation, or acylation, in particular acetylation. This improves the pharmaceutical properties of the vaccine of the present invention.

According to the present invention the peptide is coupled or fused to a pharmaceutically acceptable carrier. Preferably, the pharmaceutically acceptable carrier is one or more of KLH (Keyhole Limpet Hemocyanin), tetanus toxoid, albumin binding protein, bovine serum albumin, a dendrimer (MAP; Biol. Chem. 358:581) as well as the adjuvant substances described in Singh & O'Hagan, 1999 (specifically those in table 1 of this document) and O'Hagan & Valiante, 2003 (specifically the innate immune potentiating compounds and the delivery systems described therein, or mixtures thereof, such as low soluble aluminium compositions (e.g. aluminium hydroxide) MF59 aluminium phosphate, calcium phosphate, cytokines (e.g. IL-2, IL-12, GM-CSF), saponins (e.g. QS21), MDP derivatives, CpG oligos, IC31, LPS, MPL, squalene, D,L-alpha-tocopherol (e.g. mixed in an oil-in-water system with phosphate buffered saline), polyphosphazenes, emulsions (e.g. Freund's, SAF), liposomes, virosomes, iscoms, cochleates, PLG microparticles, poloxamer particles, virus-like particles, heat-labile enterotoxin (LT), cholera toxin (CT), mutant toxins (e.g. LTK63 and LTR72), microparticles and/or polymerized liposomes may be used). In a preferred embodiment of the invention the vaccine composition contains aluminium hydroxide. Preferably the peptides are covalently coupled or fused to the carrier.

In a particularly preferable embodiment, the at least one peptide of the inventive vaccine has an additional cystein residue added at its N-terminus and/or C-terminus and the at least one peptide is covalently coupled to the protein carrier, or a linker coupled thereto, through the additional cystein residue, preferably wherein the linker contains a maleimide group or haloacetyl group that reacts with the cystein of the peptide.

It is advantageous to provide the vaccine of the present invention together with additional excipients, for instance to increase the stability of the antibody in storage. Consequently, another preferred embodiment provides the inventive vaccine, further comprising one or more pharmaceutically acceptable excipients and/or adjuvants.

Suitable excipients are known to the person skilled in the art, for example water (especially water for injection), saline, Ringer's solution, dextrose solution, buffers, Hank solution, vesicle forming compounds (e.g. lipids), fixed oils, ethyl oleate, 5% dextrose in saline, substances that enhance isotonicity and chemical stability, buffers and preservatives. Other suitable excipients include any excipient that does not itself induce the production of antibodies in the patient that are harmful for the patient. Examples are well tolerable proteins, polysaccharides, polylactic acids, polyglycolic acid, polymeric amino acids and amino acid copolymers. This vaccine can (as a drug) be administered via appropriate procedures known to the skilled person to a patient in need thereof (i.e. a patient having or having the risk of developing the diseases or conditions mentioned herein). The patient is preferably human.

The vaccine according to the present invention is preferably formulated with an adjuvant, more preferably a low soluble aluminum composition, in particular aluminum hydroxide. Of course, also adjuvants like MF59, aluminum phosphate, calcium phosphate, cytokines (e.g. IL-2, IL-12, GM-CSF), saponins (e.g. QS21), MDP derivatives, CpG oligonucleotides, LPS, MPL, polyphosphazenes, emulsions (e.g. Freund's, SAF), liposomes, virosomes, iscoms, cochleates, PLG microparticles, poloxamer particles, virus-like particles, heat-labile enterotoxin (LT), cholera toxin (CT), mutant toxins (e.g. LTK63 and LTR72), microparticles and/or polymerized liposomes may be used.

Suitable adjuvants are commercially available as, for example, AS01B, AS02A, AS15, AS-2 and derivatives thereof (GlaxoSmithKline, Philadelphia, Pa.); CWS, TDM, Leif, aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7 or -12 may also be used as adjuvants.

Another preferred adjuvant is a saponin or saponin mimetics or derivatives, preferably QS21 (Aquila Biopharmaceuticals Inc.), which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol as described in WO 96/33739. Other preferred formulations comprise an oil-in-water emulsion and tocopherol. A particularly potent adjuvant formulation involving QS21, 3D-MPL and tocopherol in an oil-in-water emulsion is described in WO 95/17210. Additional saponin adjuvants of use in the present invention include QS7 (described in WO 96/33739 and WO 96/11711) and QS17 (described in U.S. Pat. No. 5,057,540 and EP 0 362 279 B1).

Other preferred adjuvants include Montanide ISA 720 (Seppic, France), SAF (Chiron, Calif., United States), ISCOMS (CSL), MF-59 (Chiron), the SBAS series of adjuvants (e.g., SBAS-2, AS2′, AS2, SBAS-4, or SBAS6, available from GlaxoSmithKline), Detox (Corixa), RC-529 (Corixa, Hamilton, Mont.) and other amino-alkyl glucosaminide 4-phosphates (AGPs). Further example adjuvants include synthetic MPL and adjuvants based on Shiga toxin B subunit (see WO 2005/112991).

The inventive vaccine may be administered by any suitable mode of application, e.g. intradermally (i.d.), intraperitoneally (i.p.), intramuscularly (i.m.), intranasally, orally, subcutaneously (s.c.), etc. and in any suitable delivery device (O'Hagan et al., Nature Reviews, Drug Discovery 2 (9), (2003), 727-735). The vaccine of the present invention is preferably formulated for intradermal, subcutaneous or intramuscular administration. Means and methods for obtaining respective formulations are known to the person skilled in the art (see e.g. “Handbook of Pharmaceutical Manufacturing Formulations”, Sarfaraz Niazi, CRC Press Inc, 2004).

The vaccine of the present invention is provided in injectable dosage unit form, eg as a solution, suspension or emulsion, preferably formulated in conjunction with the above-defined pharmaceutically acceptable excipients and/or adjuvants. The dosage and method of administration, however, depends on the individual patient to be treated.

Typically, the vaccine contains the one or more peptides according to the present invention each in an amount of 0.1 ng to 10 mg, preferably 10 ng to 1 mg, especially 100 ng to 100 μg or, alternatively e.g. 100 fmole to 10 μmole, preferably 10 pmole to 1 μmole, especially 100 pmole to 100 nmole.

The vaccine may also comprise typical auxiliary substances, e.g. buffers, stabilizers, etc.

The amount of peptides that may be combined with the carrier materials to produce a single dosage form will vary depending upon the host treated and the particular mode of administration. The dose of the vaccine may vary according to factors such as the disease state, age, sex and weight of the individual, and the ability of antibody to elicit a desired response in the individual. Dosage regime may be adjusted to provide the optimum therapeutic response. For example, several divided doses may be administered daily, weekly, or monthly or in other selected intervals or the dose may be proportionally reduced as indicated by the exigencies of the therapeutic situation. The dose of the vaccine may also be varied to provide optimum preventative dose response depending upon the circumstances. For instance, the peptides and vaccine of the present invention may be administered to an individual at intervals of several days, one or two weeks or even months depending always on the level of antibodies directed to the respective antigen.

As the preferred mode of administration is an administration by injection, the vaccine according to the present invention is preferably liquid or ready to be dissolved in liquid such sterile, de-ionised or distilled water or sterile isotonic phosphate-buffered saline (PBS). Preferably, 1000 μg (dry-weight) of such a vaccine comprises or consists of 0.1-990 μg, preferably 1-900 μg, more preferably 10-200 μg peptides, and optionally 1-500 μg, preferably 1-100 μg, more preferably 5-15 μg (buffer) salts (preferably to yield an isotonic buffer in the final volume), and optionally 0.1-999.9 μg, preferably 100-999.9 μg, more preferably 200-999 μg other excipients. Preferably, 1 mg of such a dry vaccine is dissolved in sterile, de-ionised/distilled water or sterile isotonic phosphate-buffered saline (PBS) to yield a final volume of 0.1-100 ml, preferably 0.5-20 ml, more preferably 1-10 ml.

According to a particularly preferred embodiment of the present invention the vaccine may comprise two or more of the following components/characteristics:

| | |
|---|---|
| Antigen (amount of | 0.1 μg to 1 mg, preferably 0.5 μg to |
| peptide per dosis) | 500 μg, more preferably 1 μg to 100 μg, |
| | net peptide |
| carrier | anything known to a person skilled in |
| | the art that is pharmaceutically and |
| | medically acceptable |
| carrier per dosis | 0.1 μg to 50 mg carrier |
| adjuvant/amount per | anything that is medically and |
| dosis | pharmaceutically acceptable |
| injection volume | anything that is medically acceptable |
| | (also depending on route of |
| | application) |
| buffer | anything that is medically and |
| | pharmaceutically acceptable |

Opn, MmpOpn and ThrOpn are involved in pathogenic processes in the human body, as described in much detail in sections above. In addition, Examples and Figures show that the vaccines and antibodies of the invention are functional. Therefore, the composition or vaccine of the present invention is preferably used in therapy, in particular in treatment and/or prevention of CVD, especially atherosclerosis, or of T2D, especially obesity-related insulin resistance.

In another aspect of the invention, a method for manufacturing the inventive antibody is provided that comprises:
expressing the antibody in cell culture
purifying the antibody.

Both expression and purification of mAbs is well-known in the art. See for instance Birch & Racher, Adv Drug Deliv Rev 2006, PMID: 16822577, for an overview of large-scale antibody production (expression and purification). Techniques comprise conventional mAb methodology, for example the standard somatic cell hybridization technique of Kohler and Milstein (1975) Nature 256: 495. Other techniques for producing mAbs can also be employed such as viral or oncogenic transformation of B lymphocytes.

In another aspect of the invention, a method for manufacturing the inventive vaccine is provided that comprises:
providing the peptides
coupling the peptides to the carrier, preferably KLH protein
optionally, adding pharmaceutically acceptable excipients.

Methods for chemical synthesis of peptides present in the inventive vaccine are well-known in the art. Of course it is also possible to produce the peptides using recombinant methods. The peptides can be produced in microorganisms such as bacteria, yeast or fungi, in eukaryotic cells such as mammalian or insect cells, or in a recombinant virus vector such as adenovirus, poxvirus, herpesvirus, Simliki forest virus, baculovirus, bacteriophage, sindbis virus or sendai virus. Suitable bacteria for producing the peptides include E. coli, B. subtilis or any other bacterium that is capable of expressing such peptides. Suitable yeast cells for expressing the peptides of the present invention include Saccharomyces cerevisiae, Schizosaccharomycespombe, Candida, Pichiapastoris or any other yeast capable of expressing peptides. Corresponding means and methods are well known in the art. Also methods for isolating and purifying recombinantly produced peptides are well known in the art and include e.g. gel filtration, affinity chromatography, ion exchange chromatography etc. Beneficially, cystein residues are added to the epitope peptides to facilitate coupling to the carrier, especially at the N- and/or C-terminus.

To facilitate isolation of said peptides, fusion polypeptides may be made wherein the peptides are translationally fused (covalently linked) to a heterologous polypeptide which enables isolation by affinity chromatography. Typical heterologous polypeptides are His-Tag (e.g. His6; 6 histidine residues), GST-Tag (Glutathione-S-transferase) etc. The fusion polypeptide facilitates not only the purification of the peptides but can also prevent the degradation of the peptides during the purification steps. If it is desired to remove the heterologous polypeptide after purification, the fusion polypeptide may comprise a cleavage site at the junction between the peptide and the heterologous polypeptide. The cleavage site may consist of an amino acid sequence that is cleaved with an enzyme specific for the amino acid sequence at the site (e.g. proteases).

The coupling/conjugation chemistry (e.g. via heterobifunctional compounds such as GMBS and of course also others as described in “Bioconjugate Techniques”, Greg T. Hermanson) in this context can be selected from reactions known to the skilled in the art. Pharmaceutically acceptable excipients are discussed in detail above and also known in the art.

The inventive antibodies are also suitable for diagnosis and/or prognosis of diseases that involve alterations in the levels of ThrOpn or MmpOpn or both in the body of the patient (as shown in FIG. 6 and Example 6). Therefore, in another aspect of the invention, a diagnostic method is provided that comprises:
providing an isolated sample of the patient, preferably a sample from blood and/or adipose tissue, especially from subcutaneous adipose tissue
using the antibody of any one of claims 1 to 10 to measure levels of ThrOpn, MmpOpn and/or the aggregate level of ThrpOpn and MmpOpn in the sample, preferably by an ELISA or Western blot
comparing these levels to levels of a healthy control population and/or to levels of the patient at an earlier time point
create a diagnosis or prognosis of a disease or condition or of the progression of said disease or condition, preferably cardiovascular disease, in particular atherosclerosis, or type-2 diabetes, in particular obesity-related insulin resistance. The patient is preferably human.

In a Western blot, the band intensity of a sample from a patient to be diagnosed with said disease or condition (i.e. a disease involving abnormal levels of cleaved osteopontin, especially obesity-related insulin resistance and/or atherosclerosis) is significantly elevated compared to the appropriate control (cf. FIG. 6 and Example 6). Typically, the band intensity is at least 25% higher, in particular 50% higher or even 75% higher compared to the appropriate control. The skilled artisan appreciates that the actual difference of the sample from a patient to be diagnosed as having said disease or condition, compared to the control, depends on many factors, such as the way of measuring antibody binding, sample treatment, etc., and can easily adapt the inventive method to said factors, including other ways of measuring antibody binding (e.g. by ELISA).

This method is not performed in or on the patient's body.

Preferably, this method is used to monitor the efficacy of any therapy described herein.

In general, peptides to raise an antibody of the present invention or peptides as components of a vaccine of the present invention can be amidated at their C-terminus or otherwise modified as known in the art. Especially the peptides with the sequences TYDGRGDSVVYG and PTVDTYDGRGDS can be amidated at their C-termini, in order to direct the antibody response to a more central part of the peptide used for immunization.

Furthermore, the antibody and vaccine of the present invention is provided in isolated form, i.e. outside of the animal and/or human body.

Herein, if not stated otherwise, the expressions osteopontin, Opn, flOpn, MmpOpn, and ThrOpn, respectively, always refer to human osteopontin, human Opn, human flOpn, human MmpOpn, and human ThrOpn, respectively.

As used herein, the term excipient is broader than the term carrier, i.e. each carrier is an excipient but not vice versa.

“Treating” as used herein means to cure an already present disease state or condition. Treating can also include inhibiting, i.e. arresting the development of a disease state or condition, and ameliorating, i.e. causing regression of a disease.

The term “preventing” as used herein means to completely or almost completely stop a disease state or condition from occurring in a patient or subject, especially when the patient or subject is predisposed to such a risk of contracting a disease state or condition.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs are disclosed herein.

Herein, the term “Ab” means antibody and the term “mAb” means monoclonal antibody.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-, yeast- or mammalian cell-display methods, and methods utilizing transgenic animals containing all or part of the human immunoglobulin loci, such methods and other exemplary methods for making monoclonal antibodies being described herein.

For the present invention the terms “pharmaceutical preparation” and “pharmacological composition” are used interchangeably and refers to a composition that is intended and suitable for delivery to human individuals. Such preparations or compositions have been manufactured according to GMP (good manufacturing practices) and are sufficiently sterile and packaged to comply with the prerequisites necessitated by the EMA and FDA, especially by the EMA.

The invention is further described by the following examples and the drawing figures, yet without being limited thereto.

### SHORT DESCRIPTION OF THE FIGURES

FIG. 1: Immunoreactivity of vaccine-induced sera against either full length Opn (flOpn), thrombin cleaved Opn (ThrOpn), or metalloproteinase cleaved Opn (MmpOpn). Immunoreactivity was tested using ELISA. (A) Vaccines containing peptide sequences SEQ ID NO: 1-5 predominantly elicit sera directed against ThrOpn. (B) Vaccines containing peptide sequences SEQ ID NO: 6-9 predominantly elicit sera binding to MmpOpn. Although peptide sequence SEQ ID NO: 10 ends with the same amino acids as peptides SEQ ID NO: 6-9, all of them representing the Mmp-cleavage site, the vaccine containing SEQ ID NO: 10 induces an immune serum that recognises all Opn variants tested. (C) Vaccines containing peptide sequences SEQ ID NO: 11-14 elicit sera that bind all Opn variants tested.

FIG. 2: Assessment of functional activity of vaccine induced antibodies. (A) Cell adhesion induced by full length Opn can just be blocked using sera, elicited by SEQ ID NO: 12 or 14 containing vaccines that show binding to all Opn variants including flOpn. All other sera do not show this inhibitory potential. Cell adhesion induced by either ThrOpn (B) or MmpOpn (C) can be blocked by sera specific for either ThrOpn (elicited by SEQ ID NOs: 1 or 2) or MmpOpn (elicited by SEQ ID NOs: 7 or 8) and by sera induced by vaccines containing SEQ ID NO: 12 or 14 that show pan reactivity against all Opn variants tested. Control sera do not show any effect on cell adhesion in all cases.

FIG. 3: Immunoreactivity of mAbs against either full length Opn (flOpn), thrombin cleaved Opn (ThrOpn), or metalloproteinase cleaved Opn (MmpOpn). Immunoreactivity was tested using ELISA. (A) mAb 4-4-2 shows exclusively binding to ThrOpn. (B) and (C) mAbs 7-5-4 and 9-3-1 show a minor cross-reactivity towards ThrOpn but are highly reactive towards MmpOpn. (D) 21-5-4 mAb binds all Opn forms but is more reactive towards cleaved Opn variants.

FIG. 4: Immunoreactivity of mAbs in Western Blot analysis against either flOpn (lane 1-4), ThrOpn (lane 5-6), or MmpOpn (lane 7-8). (A) mAb 4-4-2 shows a weak but exclusive binding to ThrOpn. (B) and (C) mAbs 7-5-4 and 9-3-1 show binding to both cleaved Opn forms but are clearly more reactive towards MmpOpn. (D) 21-5-4 mAb binds all Opn forms but is more reactive towards cleaved Opn variants. (E) Used marker is depicted. (F) Loading scheme.

FIG. 5: Assessment of functional activity of mAbs. (A) Cell adhesion induced by ThrOpn can just be blocked by mAb 21-5-4 mAb 4-4-2 although specific for ThrOpn does not appear to have inhibitory capacities in this setting. (B) Cell adhesion induced by MmpOpn can be blocked by mAbs more reactive towards MmpOpn such as mAb 7-5-4 and 9-3-1 and by the pan-reactive mAb 21-5-4.

FIG. 6: Determination of cleaved OPN in human adipose tissue (AT). (A) Subcutaneous AT lysates were immunoblotted using mAb 9-3-1. (B) Quantification of the 25 kD bands related to b-actin of all lean (n=6) and obese (n=6) donors investigated. The diagram shows mean+/−SE. **, p<0.01 (Student's T-Test).

FIG. 7: Graphical representation of the CDR loop sequencing results of mAb 4-4-2. (A) CDR loops of V_{H} region (B) CDR loop of V_{L} region. Representation/IMGT numbering system according to Lefranc et al. (Nucleic Acids Research 1999, PMID: 12477501). Blue shaded circles are hydrophobic (non-polar) residues in frameworks 1-3 at sites that are hydrophobic in the majority of antibodies. Yellow shaded circles are proline residues. Squares are key residues at the start and end of the CDR. Red amino acids in the framework are structurally conserved amino acids.

FIG. 8: Graphical representation of the CDR loop sequencing results of mAb 7-5-4 (and identical clone mAb 9-3-1). (A) CDR loops of V_{H} region (B) CDR loop of V_{L} region. Representation/IMGT numbering system according to Lefranc et al. (Nucleic Acids Research 1999, PMID: 12477501). Blue shaded circles are hydrophobic (non-polar) residues in frameworks 1-3 at sites that are hydrophobic in the majority of antibodies. Yellow shaded circles are proline residues. Squares are key residues at the start and end of the CDR. Red amino acids in the framework are structurally conserved amino acids.

FIG. 9: Graphical representation of the CDR loop sequencing results of mAb 21-5-4. (A) CDR loops of V_{H} region (B) CDR loop of V_{L} region. Representation/IMGT numbering system according to Lefranc et al. (Nucleic Acids Research 1999, PMID: 12477501). Blue shaded circles are hydrophobic (non-polar) residues in frameworks 1-3 at sites that are hydrophobic in the majority of antibodies. Yellow shaded circles are proline residues. Squares are key residues at the start and end of the CDR. Red amino acids in the framework are structurally conserved amino acids.

### EXAMPLES

### Material and Methods

### Active Vaccination Approach

Female BALB/c mice (6-8 weeks) were primed and boost-immunized four times in biweekly intervals with KLH-conjugated peptide vaccines (200 μl subcutaneously in phosphate buffer pH 7.4). Aluminum hydroxide was used as an adjuvant. Six mice were used for injection with the respective KLH-conjugated peptide vaccine. Experiments were repeated and representatives thereof are shown below. Antibody titers of the mouse sera were analyzed by the Enzyme Linked Immunosorbent Assay (ELISA). Titers were calculated as the sera dilution giving half-maximal binding (i.e. ODmax/2) and the median titers of 5 to 6 mice per group are presented. The functional activity of the induced antibodies was assessed by the glucuronidase enzyme release assay.

Monoclonal Antibody Production

Antigen for Immunization

The mice were injected with conjugates, which consist in peptides coupled to the carrier protein KLH (MP Biomedicals) through a linker (N-Methylpyrrolidon, NMP) containing a maleimide group. We used three different peptides: SEQ ID No. 2 (Sequence SVVYGLR-COOH), SEQ ID No. 7 (Sequence GDSVVYG-COOH) and SEQ ID No. 12 (Sequence C-TYDGRGDSVVYG-CO-NH2). Sequence C-SVVYGLR-COOH was used to induce and finally produce antibodies specifically targeting ThrOpn, whereas sequence C-GDSVVYG-COOH was used aiming to produce antibodies specifically binding the MmpOpn. In contrast sequence C-TYDGRGDSVVYG-CO-NH2 was used for immunization aiming at inducing antibodies binding to the RGDSVVYG motif and thus specific for both ThrOpn and MmpOpn.

The conjugation of the peptide occurred through binding of the linker to the SH-group of the N-terminal cystein and is a two-step process. First, KLH was maleoylated; 1 mg of the linker (50 mg/ml NMP) was added to 1 ml of the KLH solution (10 mg/ml in 0.1 mM NaHCO3, pH=8.3) and incubated at room temperature (RT) for 1h. Next, the KLH-linker solution was desalted using a PBS equilibrated Sephadex G50 column (1.5×14 cm). In the second step, the maleoylated KLH was coupled to the peptide; 100 μl peptide solution (10 mg/ml in aqua bidest) was mixed to 1 ml of the maleoylated KLH solution (2 mg/ml in PBS) and incubated for 2h at RT. To block maleimide groups which do not react, 2-mercaptoethanol was added (until a concentration of 10 nM) to the solution and incubated overnight at 4° C. The conjugate was then dialyzed against PBS at 4° C. (three buffer changes, the molecular weight cut-off was set at 10.000).

Antigen for Analyses

To exclude NMP- and KLH-specific antibodies in mice serum and in the hybridomas supernatant, the peptides used for respective ELISA analyses (see “Immunization” and “Fusion of splenocytes with myeloma cells”), a distinct linker (SMPH, Succinimidyl-6-[(β-maleimidopropioamido) hexanoat], Sigma) -carrier protein (BSA, Sigma)) combination was used, according to the protocol described above.

Immunization

Female 8 week-old BALC/c mice (Janvier, France) were immunized with the conjugates by intraperitoneal injections during a period of 39 days. Serum samples were collected during the immunization time and tested in ELISA as a control for successful antibody induction.

Fusion of Splenocytes and Myeloma Cells

The hybridoma cells producing antibodies against the three Osteopontin peptides were generated by fusing splenocytes with myeloma cells according to the following protocol. In general, cells were cultured in complete DMEM medium (PAN Biotech) supplemented with antibiotics (10000 L.E penicillin, 10000 μg/ml Streptomycin, 25 μg/ml Amphotericin, 100×, PAA), 2-mercaptoethanol (Sigma), L-Glutamin (100×, PAA), stable Glutamin (100×, PAA), HT-supplement (50×, GIBCO/BRL), MEM non-essential amino acids (100×, PAA) and 10, 15 or 20% FCS (PAA). The spleens of the immunized mice were removed and processed to a single cell suspension using a homogenizer and a cell strainer. The myeloma cells SP2/0-Ag14 (SP2/0) were ordered from the “Deutsche Sammlung von Mikroorganismen and Zellkulturen (DSMZ)”, cultured in complete DMEM medium (10% FCS) and tested regularly for mycoplasma contamination. The splenocytes and the myeloma cells SP2/0 were washed with DMEM and fused with polyethylenglycol 3350 (1 ml 50% w/v, Sigma). The generated hybridomas were resuspended in complete DMEM medium (20% FCS) and Aminopterin (50×, Sigma) (HAT medium) and seeded in 96-well tissue culture plates (Corning Costar) on peritoneal feeder cells. The hybridomas were incubated for 10 days at 37° C., 5% CO2.

ELISA for screening of mice sera and hybridoma supernatants ELISA plates (PAA, Cat# PAA38096X) were coated with 4 μg/ml of peptide-BSA conjugates (see table below) in 100 mM NaHCO3 pH=9.6. The plates were then washed with TBS (10 mM Tris, 200 mM NaCl, pH=7.8) and 0.01% Triton X-100 and blocked with 2% FCS (v/v) in TBS. Hybridoma supernatant (undiluted) and sera (1:100) were diluted in blocking buffer an applied on coated plates. Cell supernatant of SP2/0 cells was used as negative control. Plates were then washed and bound antibodies were detected with an AP-conjugated goat anti-mouse IgG antibody (Sigma). 4-Nitrophenylphosphat (2 mM in 5% diethanolamine, 1 mM MgCl2, Fluka) was used as a substrate for the alkaline phosphatase (AP). The absorbance was read at 405 nm using a microplate reader (Dynex Opsys MR). Antibody titer in mice serum was determined by serum titration. For screening of the hybridomas supernatants, a clone was declared to be positive when the signal was twice as high as the mean ELISA plate value at 405 nm.

All of the following peptides were synthesised with an additional cysteine residue at the N-terminus for coupling

| | |
|---|---|
| Peptides used for | Peptides used for ELISA |
| immunization (coupled to KLH) | (coupled to BSA) to screen |
| | immunoreactivity of sera and |
| | hybridoma supernatants |
| C-SVVYGLR-COOH (SEQ ID NO: 2) | C-SVVYGLR-COOH (SEQ ID NO: 2) |
| | C-VYGLRSK-COOH (SEQ ID NO: 51) |
| | C-GDSVVYG-COOH (SEQ ID NO: 7) |
| C-GDSVVYG-COOH (SEQ ID NO: 7) | C-GDSVVYG-COOH (SEQ ID NO: 7) |
| | C-SVVYGLR-COOH (SEQ ID NO: 2) |
| | C-YDGRGDS-COOH (SEQ ID NO: 52) |
| C-TYDGRGDSVVYG-CO-NH2 | C-TYDGRGDSVVYG-CO-NH2 (SEQ ID |
| (SEQ ID NO: 12) | NO: 12) |
| | C-TYDGAAASVVYG-CO-NH2 (SEQ ID |
| | NO: 53) |
| | C-TAAARGDAAAYG-CO-NH2 (SEQ ID |
| | NO: 54) |

Selection Phase of Antibody-Producing Clones

The cells tested positive in ELISA were transferred into a 48-well plate and cultured for a couple of days. In this time period, the supernatant were tested again by ELISA on reactivity with the respective relevant peptides. The selection phase was short to avoid an overgrown of unspecific clones.

Cloning

After the selection phase, the first cloning was performed with the main aim to separate antibody-producing from non-producing cells. A second cloning ensured that the clones selected were monoclonal. In both cases, cloning was performed by limited dilution and cells transferred in a 24-well plate. After 6-8 days, the monoclonal cell growth was analyzed under the microscope. After 3 more days, the supernatants were analyzed by ELISA. The subclones showing the best growth and ELISA signals were selected for the second cloning and further for cryopreservation. The subclones supernatants were tested for mycoplasma contamination (Greiner Bio-One, Frickenhausen, Germany) and the isotype of the monoclonal antibody was determined by a commercially available kit (Serotec).

Monoclonal Antibody Sequence Determination

Monoclonal antibody sequencing was performed by Fusion Antibodies Ltd (Springbank Industrial Estate, Pembroke Loop Road, Belfast, N. Ireland, BT17 0QL)

mRNA was extracted from the hybridoma cell pellets on Oct. 10, 2013. Total RNA was extracted from the pellets using Fusion Antibodies Ltd in-house RNA extraction protocol.

RT-PCR

cDNA was created from the RNA by reverse-transcription with an oligo(dT) primer. The cDNA was S.N.A.P purified and the tailing of cDNA by TdT, were carried out using the 5′ RACE kit. PCR reactions were set up using AAP and variable domain reverse primers to amplify both the VH and VL regions of the monoclonal antibody DNA.

The VH and VL products were cloned into the Invitrogen sequencing vector pCR2.1 and transformed into TOP10 cells and screened by PCR for positive transformants. Selected colonies were picked and analyzed by DNA sequencing on an ABI3130xl Genetic Analyzer, the result may be seen below.

Cloning, Generation and Production of Recombinant Human Opn Proteins

3 recombinant forms of Opn including indicated Tags (Strep, 6×His) were expressed and purified:

a) Strep—314 AA (full length Opn)—6×His

b) 6×His—166 AA (N-terminal MMP-cleaved Opn)

c) 6×His—168 AA (N-terminal thrombin-cleaved Opn)

As a template the cDNA clone BC017387 (Thermo Scientific) was used.

The DNA sequences were amplified using the same forward primer (5′-AGCGGCTCTTCAATGATACCAGTTAAACAGGCTGATTC-3′; SEQ ID NO: 42) and following reverse primers:

a) Strep—314 AA (5′-AGCGGCTCTTCTCCCATTGACCTCAGAAGATGCACT-3′; SEQ ID NO: 43),

b) 6×His—166 AA (5′-AGCGGCTCTTCTCCCCTATCCATAAACCACACTATCACC-3′; SEQ ID NO: 44) (includes stop codon after 166 AA for solely N-terminal tagged forms)

c) 6×His—168 AA (5′-AGCGGCTCTTCTCCCCTACCTCAGTCCATAAACCACAC-3′; SEQ ID NO: 45) (includes stop codon after 168 AA for solely N-terminal tagged forms).

The amplicons were cloned using the StarGate Entry Cloning system (IBA, Gottingen) into the pENTRY-IBA51 plasmid and then subcloned into either the pCSG-IBA142 or the pCSG-IBA144 plasmid (all IBA), introducing an N-terminal 6× Histidine-tag, N-terminal OneStrep®-tag, C-terminal 6× Histidine-tag, respectively, following the manufacturer's instructions.

Transient expression in the HEK293 c18 cell line (ATCC, CRL-10852) was induced by transfection using Lipofectamine 2000 (Life Technologies, Carlsbad) followed by purification with Ni-NTA Resin (Merck, Darmstadt) or Strep-Tactin (IBA, Gottingen) packed gravity flow columns. Purified proteins were analyzed by sodium dodecyl-sulfate polyacrylamide gel electrophoresis (SDS-PAGE) and dialyzed against PBS.

ELISA for cross-reactivity of monoclonal antibodies with recombinant Opn proteins

ELISA plates (Nunc, Cat#439454) were coated with 1 μg/ml recombinant human Opn proteins (home-made or from PeproTech, Cat#120-35 as control) in 100 mM NaHCO3 pH=9,2 and blocked with 1% BSA in 1×PBS. Bound mouse anti-human monoclonal antibodies (BioGenes, 1 μg/ml) and the mouse serum used as positive control were detected using a HRP-conjugated goat anti-mouse IgG antibody (Jackson Laboratory, Cat#115-035-068, 0.2 μg/ml). ABTS (0.68 mM ABTS in 0.1 M citric acid, pH=4.3) and 0.1% hydrogen peroxide was used as a substrate for the horse radish peroxidase (HRP). The substrate reaction was stopped by adding 1% SDS solution to the wells. The absorbance was read at 405 nm using a microplate reader (TECAN Sunrise).

Gel electrophoresis and Western Blot For gel electrophoresis, 4-20% Criterion TDX precast gels (BioRad, Cat#567-1095) were used. Recombinant human Opn proteins (home-made or from PeproTech, Cat#120-35) were denatured at 70° C. for 10 min in a reducing loading buffer (4×LDS-Buffer, Invitrogen, Cat# NP0007 supplemented with 0.1% (v/v) β-Mercaptoethanol). The amount of loaded protein per well was 0.6 μg. The Precision Plus Protein Dual (Biorad, Cat#161-0374) was used as protein ladder and a 1× Tris/Glycin/SDS buffer as running buffer (Biorad, Cat#161-0732). The protein were transferred to a 0.2 μm nitrocellulose membrane using the Transfer Turbo blot transfer pack (Biorad, Cat#170-4159). The membrane was blocked with a commercially available blocking solution (Thermo scientific, #37515). Antibodies were diluted in 1:10 blocking solution. Membranes were first incubated with the mouse anti-human monoclonal antibodies (BioGenes, 1 μg/ml) for 1h and washed with 0.1% PBST. For detection, a HRP-conjugated IgG antibody (Southern Biotech, Cat#1034-05, 1:40.000) was incubated for 45 min. Membranes were then washed with washing buffer and dH2O. Membranes were finally incubated with the substrate (ECL Clarity Western, Biorad #170-5061) for 5 min. Image acquisition was performed using LabWorks software 4.6.

Functional Assay—Adhesion Assay

Microplates with V-shaped bottom (Greiner, Cat#651161) were coated with 0, 10 or 30 nM recombinant human Opn proteins (1h, 37° C.) and blocked with 1% BSA/1×PBS (1h, 37° C.). After washing with 1×PBS, 5 μg/ml blocking antibodies were added to the wells (1h at 37° C.). Mouse anti-human monoclonal antibodies (BioGenes) and an IgG1 isotype control (Sigma, Cat# M9269) were used. Next, plates were washed and 20.000 of previously CMFDA-labeled HEK293 cells were added to the wells (30 min, 37° C.). Shortly, HEK293 cells were harvested with 0.02% EDTA (Versene, GIBCO, Cat#15040) and resuspended in DMEM without phenol red (Sigma, Cat# D5921) supplemented with 10% FCS, 1% penicillin/streptomycin (GIBCO, Cat#15140) and 4 mM L-Glutamine (GIBCO, Cat#25030). For labeling, 1.5 μM CMFDA cell tracker (Molecular Probes, Cat# C7025) were added to the HEK293 cells (30 min, 37° C., 500 rpm). Cells were washed twice with DMEM without phenol red before being added to the wells. After incubation of the cells on the plates, plates were centrifuged for 5 min at 100 g. As non-adherent cells move to the bottom of the V-shaped wells, they were quantified by measuring the fluorescence intensity through bottom reading (Genios microplate reader, Excitation 485 nm, Emission 535 nm).

Surface Plasmon Resonance (SPR)

Binding analyses were performed on a Biacore 2000. The different biotinylated peptide ligands representing the different Opn products were immobilized on a streptavidin chip in one flow cell respectively, with a flow rate of 30 μl/min. The fourth flow cell is a reference cell without peptide. The peptides were dissolved in a DMSO solution and further diluted in HBS buffer (0.01M Hepes, 0.1M NaCl, 0.004M EDTA, 0.05% P20, pH=7.4). After immobilization, the chips in each flow cell were saturated with 1 μM D-Biotin. As a control, an unspecific serum was injected through all cells. The flow rate of the antibodies was 30 μl/min at a concentration of 2 μg/ml, volume injected 100 μl. The dissociation time was set at 500 sec. The chips were regenerated with 60 μl of 50 mM HCl. Off-rate values and KD values were calculated using the Biocore SPR software.

The inventive mAb as diagnostic tool—determination of OPN concentration in human samples (see also Example 6)

Subcutaneous adipose tissue (AT) biopsies were obtained from severely obese (BMI40 kg/m2) or age- and sex-matched lean to overweight controls (BMI≦30) nondiabetic patients [fasting plasma glucose <126 mg/dL and 2-h plasma glucose after a 75-g oral-glucose-tolerance test (OGTT)<200 mg/dL] between 20 and 65 y of age who underwent elective bariatric or other laparoscopic abdominal surgery. Patients were excluded in cases of acute illness within the past 2 wk; known diabetes mellitus or use of antidiabetic medication; acquired immunodeficiency (HIV infection); hepatitis or other significant liver disease; severe or untreated cardiovascular, renal, or pulmonary disease; untreated or inadequately treated clinically significant thyroid disease; anemia; active malignant disease; inborn or acquired bleeding disorder, including warfarin treatment; pregnancy or breastfeeding.

50 mg of frozen AT homogenized in 210 μl of lysing-buffer 210 μl of lysing-buffer containing 20 mM Tris, 140 mM NaCl, 1% Triton-X, complete-protease inhibitor cocktail (Roche) and sodium-orthovanadate (1 mM) adjusted to pH 7.4. Subsequently samples were boiled in reducing sample loading buffer, at 95° C. for 10 min and centrifuged at 10 k U/min at 4° C. The fluid phase under the fatty layer was taken, repeatedly centrifuged and protein concentration was measured using 600 nm protein assay kit (Thermo Scientific). 10 μg of proteins per sample were separated on SDS-PAGE using 12% polyacrylamide gels and blotted onto a nitrocellulose membrane according to standard Western blot procedures. OPN fragment at 25 kDa was detected using mAb CMI 9-3 at 20 μg/ml and HRP-labeled goat anti-mouse (BioRad #170-6516). Anti-b-actin (Novus Biologicals #AC-15) served as loading control. Blots were visualized and band intensities quantified using a Fusion-FX imaging instrument (Peqlab) and ImageJ software (ImageJ, U. S. National Institutes of Health).

Active Vaccination

### Example 1

Induction of an antibody immune response specifically binding to the thrombin-cleaved or MMP3/7/9-cleaved form of Opn as well as induction of an antibody response binding the 159RGD161 region in the context of the cryptic domain and thus binding all forms of Opn (full length, thrombin-cleaved, and MMP3/7/9-cleaved Opn)

In this study mice were immunized with conjugate vaccines containing one of the antigenic peptides as listed in Table 1. The aim of this study was to identify antigenic peptides that have the capacity to induce an antibody response specifically binding to the truncated forms of Opn, either the thrombin-cleaved or the MMP3/7/9-cleaved form of Opn. Furthermore, the study was aimed at identifying antigenic peptides that induce an antibody response binding to the 159RGD161 motive in the context of the cryptic domain of Opn, thus being Opn specific but not differentiating between different forms of Opn (full length, thrombin-cleaved, and MMP3/7/9-cleaved Opn).

In order to identify peptides able to induce an antibody response specifically targeting the neoepitope generated by thrombin-cleavage, peptides that differ in their length (SEQ ID No. 1-5) were used for immunization. All these peptides end at their C-termini with the Arginin (R) that represents the thrombin-cleavage site. These peptides were not amidated at their C-termini.

In order to identify peptides able to induce an antibody response specifically targeting the neoepitope generated by MMP3/7/9-cleavage, again peptides that differ in their length (SEQ ID No. 6-10) were used for immunization. All these peptides end at their C-termini with the Glycine (G) that represents the MMP-cleavage site. These peptides were not amidated at their C-termini.

In order to identify peptides able to induce an antibody response specifically targeting the RGD region of Opn (located at position 159-161) in the context of flanking amino acids (including the cryptic domain) of Opn peptides SEQ ID No. 11-14 were used for immunization. These peptides have been designed in the way that the antigenic peptides used for immunization walk over the RGD motive. These peptides were amidated at their C-termini, in order to direct the antibody response to a more central part of the peptide used for immunization.

TABLE 1
Peptide Sequences of Opn used in the present
invention for an active peptide-base conjugate
vaccine to induce an antibody immune-response
targeting Opn. Peptides SEQ ID No. 1-
SEQ ID No. 10 have a free carboxyl end (COO⁻)
whereas the C-terminal end of peptides SEQ ID No.
11-14 are preferably amidated (COO-NH₂) (and were
amidated in the experiments described herein)

| Sequence | Amino acid | |
|---|---|---|
| identification | Opn | |
| number | position of | Sequence |
| SEQ ID No: 1 | 163-168 | VVYGLR |
| SEQ ID No: 2 | 162-168 | SVVYGLR |
| SEQ ID No: 3 | 161-168 | DSVVYGLR |
| SEQ ID No: 4 | 159-168 | RGDSVVYGLR |
| SEQ ID No: 5 | 157-168 | DGRGDSVVYGLR |
| SEQ ID No: 6 | 161-166 | DSVVYG |
| SEQ ID No: 7 | 160-166 | GDSVVYG |
| SEQ ID No: 8 | 159-166 | RGDSVVYG |
| SEQ ID No: 9 | 157-166 | DGRGDSVVYG |
| SEQ ID No: 10 | 155-166 | TYDGRGDSVVYG |
| SEQ ID No: 11 | 157-168 | DGRGDSVVYGLR |
| SEQ ID No: 12 | 155-166 | TYDGRGDSVVYG |
| SEQ ID No: 13 | 153-164 | VDTYDGRGDSVV |
| SEQ ID No: 14 | 151-162 | PTVDTYDGRGDS |

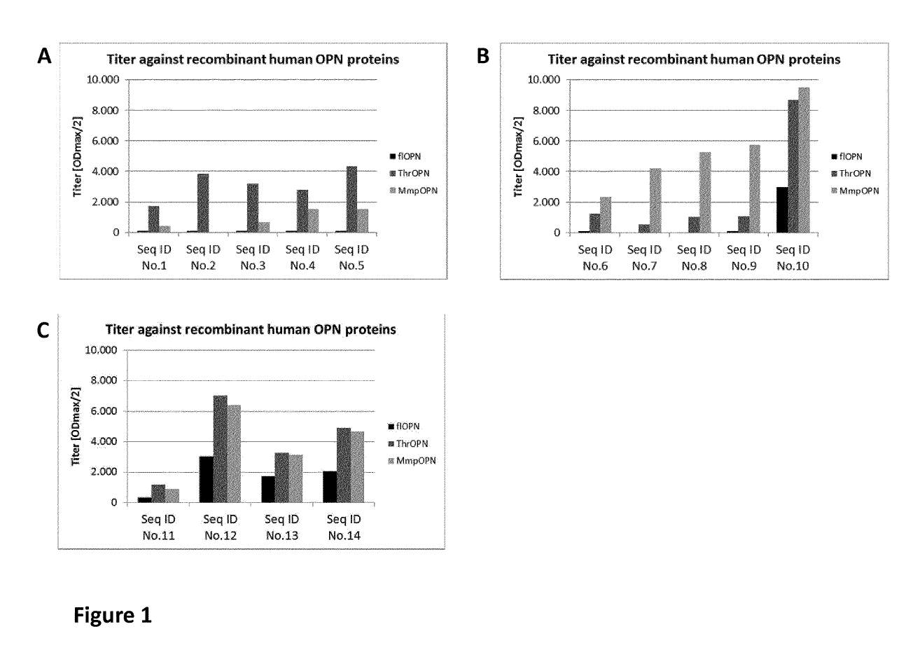

Mice were immunized with the indicated peptides and the median titers of the induced antibodies against different recombinantly produced forms of Opn (full length [flOpn], truncated Opn ending with the thrombin cleavage site 1-0pn-168 [ThrOpn], truncated Opn ending with the MMP cleavage site 1-0pn-166 [MmpOpn]) are shown in FIG. 1. Although all tested peptides were able to induce antibodies which bind to the injected peptide (data not shown) with the same magnitude, sera elicited by these peptides differ very much in terms of their reactivity against flOpn, ThrOpn, and MmpOpn.

Surprisingly, peptides SEQ ID No. 1 (although the magnitude of the immune response was lower compared to SEQ ID No. 2 and SEQ ID No. 3), SEQ ID No. 2 and SEQ ID No. 3 induced an immune response almost specifically for the thrombin-cleaved form of Opn. Whereas, SEQ ID No. 4 and SEQ ID No. 5-induced antibodies appeared to bind also to MmpOpn.

In contrast, peptides SEQ ID No. 7, SEQ ID No. 8, and SEQ ID No. 9 induce an antibody response targeting the MMP-cleaved form of Opn with high specificity and high magnitude (FIG. 1B). This is not the case when sera were analyzed that have been elicited by the other proteins.

Interestingly, peptides SEQ ID No. 10, SEQ ID No. 12, and SEQ ID No. 14 induced sera that react against all forms of Opn (FIG. 1C). Importantly, the elicited sera are specific for Opn and thus binding of antibodies require the amino acids 159RGD161 in the context of flanking Opn specific amino acids.

Importantly, vaccines used as controls did not induce an antibody response binding to one of the Opn forms (data not shown).

### Example 2: Functional Evaluation of Vaccine-Induced Immune Sera

In order to evaluate the potential of vaccine-elicited sera (as described in Example 1) to block the activity of the different forms of Opn (full length, Thr-cleaved, or MMP-cleaved Opn) functional adhesion assays as described in detail in the Material and Methods section were performed. Shortly, 96 well plates were either coated with recombinantly produced full length Opn, with truncated recombinant Opn protein ending with the thrombin-cleavage site (ThrOpn or 1-0pn-168) or with truncated recombinant Opn protein ending with the MMP3/7/9-cleavage site (MmpOpn or 1-Opn-166). Subsequently, sera derived from immunized animals as described in Example 1 and HEK293 cells labelled with a fluorescent dye were added to plates. Adhesion was measured and calculated as described in the Material and Methods section.

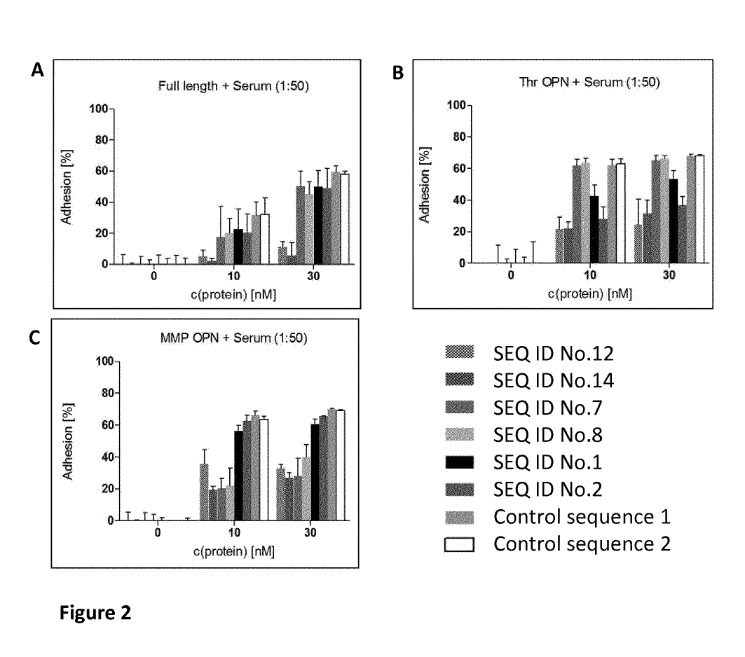

As can be seen in FIG. 2 sera elicited by peptide sequences SEQ ID No. 1 and SEQ ID No. 2, which have been shown to specifically target the Thr-cleaved form of Opn (FIG. 1A), just block the adhesion of HEK293 cells to ThrOpn (FIG. 2B) without interfering with the adhesion of HEK293 cells to the other Opn forms (FIGS. 2A and 2C) confirming the specificity of SEQ ID No. 1 and SEQ ID No. 2 induced sera (FIG. 1). Importantly, inhibition of the adhesion of HEK293 cells to ThrOpn coated plates was less pronounced when sera elicited by SEQ ID No. 1 were used compared to sera elicited by the peptide SEQ ID No. 2. This is in line with the reactivity of these induced sera against Thr-Opn (FIG. 1A).

In contrast to SEQ ID No. 1 and SEQ ID No. 2, SEQ ID No. 7 and SEQ ID No. 8, shown to specifically targeting the MmpOpn (FIG. 1B), block the adhesion of HEK293 cells just on plates that have been coated with the truncated Opn ending with the MMP-cleavage site (MmpOpn) (FIG. 2C). These data again confirm the specificity of induced sera for MmpOpn.

SEQ ID No. 12 and SEQ ID No. 14, shown to bind all forms of Opn (FIG. 1), have the capacity to block the adhesion of HEK293 cells to all different Opn versions FIG. 2A-2C), confirming again their pan-reactivity.

As controls, sera elicited by irrelevant peptide sequences, were used (Control sequence 1 and 2). As expected these sera did not interfere with the adhesion of HEK293 cells to each Opn version.

Monoclonal Antibody Generation and Evaluation of these mAbs

### EXAMPLE 3: Generation and Purification of Monoclonal Antibodies as Well as Determination of Binding Characteristics Against Different Opn Products by ELISA and Western Blot

In order to generate mAbs specifically targeting and thus distinguishing different Opn products (full length, ThrOpn and MmpOpn) mice were immunized with different peptide sequences shown to induce specific antibody responses (FIG. 1 and FIG. 2)

As outlined in Table 2, in order to generate antibodies specifically binding the Thr-cleaved Opn product SEQ ID No. 2 was used for immunization. In order to generate antibodies specifically binding the MMP-cleaved Opn product SEQ ID No. 7 was used for immunization, and in order to generate antibodies binding all Opn products (irrelevant whether full length or cleaved) SEQ ID No. 12 was used for immunization.

Hybridoma cell lines producing monoclonal antibodies were generated, selected and purified as described in the Material and Method section. A great number of mAb producing hybridoma cell lines were judiciously screened and analyzed and finally four different antibody producing hybridoma cell lines were selected as listed in Table 2. Monoclonal Abs were purified from the cell culture supernatant by affinity purification on a protein A column and characterized and classified as described in Table 2.

TABLE 2
Characteristics of the monoclonal antibodies

| Name of | Peptide used for | Isotype | Isotype |
|---|---|---|---|
| clone | immunization | Heavy chain | Light chain |
| 4-4-2 | SEQ ID No. 2 | IgG₁ | k |
| | SVVYGLR-COOH | | |
| | Thrombin cleavage site | | |
| | specific | | |
| 7-5-4 | SEQ ID No. 7 | IgG₁ | k |
| | GDSVVYG-COOH | | |
| | Mmp cleavage site | | |
| | specific | | |
| 9-3-1 | SEQ ID No. 7 | IgG₁ | k |
| | GDSVVYG-COOH | | |
| | Mmp cleavage site | | |
| | specific | | |
| 21-5-4 | SEQ ID No. 12 | IgG₁ | k |
| | TYDGRGDSVVYG-CO-NH2 | | |
| | RGD site in Opn | | |
| | context | | |

In order to characterize the binding capacity of mAbs in more detail ELISA studies and Western Blot analyses were performed.

First, the ability of the different mAbs to specifically recognize different Opn products (full length or truncated Opn fragments) was tested using ELISA.

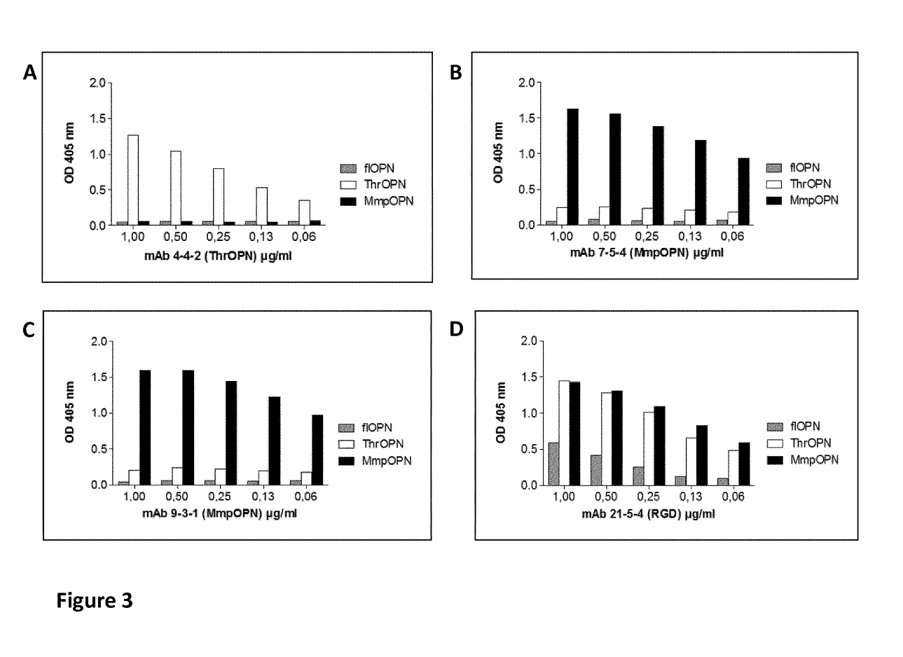

As shown in FIG. 3 the specificity of individual monoclonal antibodies for native full length and protease-cleaved Opn was determined. FIG. 3A shows that mAb 4-4-2 specifically interacts with ThrOpn, whereas mAb 7-5-4 and mAb 9-3-1 specifically bind MmpOpn (FIGS. 3B and 3C, respectively). Reactivity of mAb 21-5-4 that was elicited by SEQ ID No. 12, known to direct the antibody response towards the RGD region, is shown in FIG. 3D. This mAb appears to bind all Opn products although reactivity against cleaved Opn forms is more pronounced.

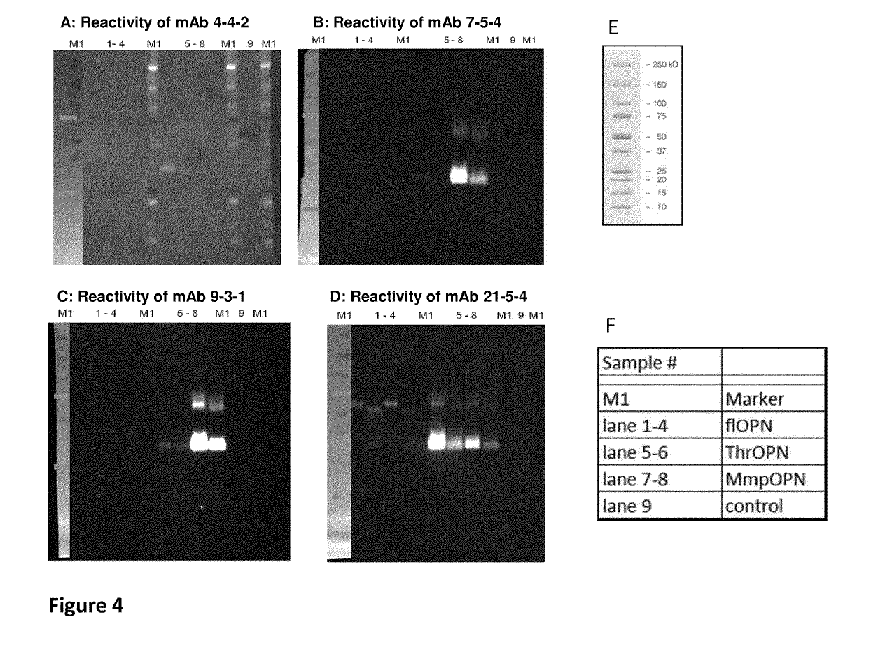

In a next set of experiments, the capacity of the different mAbs to distinctly bind protease-cleaved Opn fragments and/or full length Opn protein was determined by Western-Blot analyses (FIG. 4). Monoclonal Ab 4-4-2 reacts with low signal intensity against ThrOpn (FIG. 4A, lane 5 and 6), whereas mAbs 7-5-4 and mAb 9-3-1 specifically interact with MmpOpn with high intensity (FIGS. 4B and 4C, respectively; lane 7 and 8). As expected from ELISA data mAb 21-5-4 stains all Opn forms (FIG. 4D; full length Opn—lane 1-4, proteolytically cleaved Opns—lane 5-8). Again, full length Opn was detected with lower intensity on Western Blots than cleaved Opn forms. Since recombinantly produced full length Opn proteins applied to the lanes 1-4 contain different tag systems, these proteins differ in their size and thus appear on different positions on the gel and/or blot. FIG. 4E represents the marker M1 used in these experiments and FIG. 4F depicts how the different Opn products were applied to the gel.

### Example 4: Functional Evaluation of Selected mAbs 4-4-2, 7-5-4, 9-3-1, and 21-5-4 Using Adhesion Assay

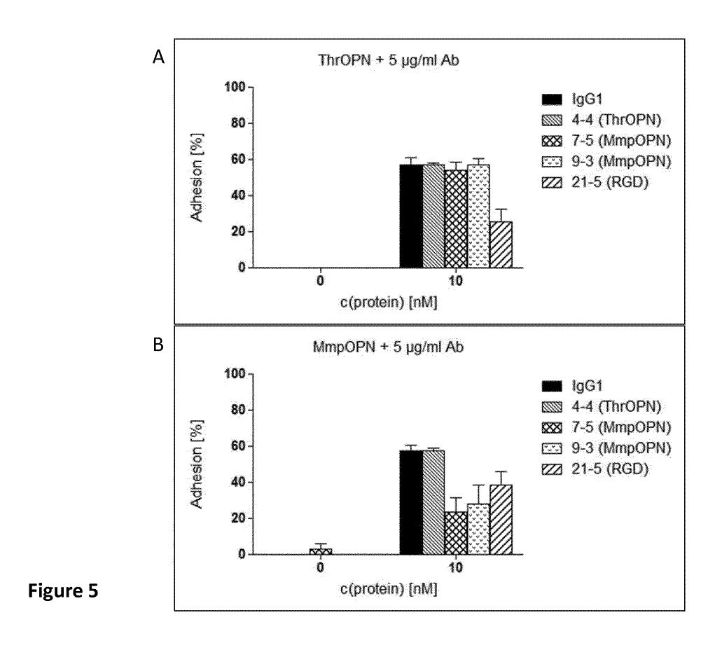

In a next set of experiments further analyses were performed in order to characterize the functional activity of the antibodies in terms of inhibiting the adhesion of HEX293 cells to respective Opn fragments in the described cell-based assay. ThrOpn and MmpOpn were used for this purpose. FIG. 5A depicts the ability of monoclonal antibodies to inhibit the adhesion of HEK293 cells to Thrombin-cleaved Opn (ThrOpn). Although mAb 4-4-2 has been shown to specifically interact with ThrOpn (FIGS. 3 and 4) it has not the capacity to block ThrOpn in this functional assay (FIG. 5A). This discrepancy between binding of the antibody to Opn in ELISA and Western Blot and missing functional activity in the cell based assay can be explained by its rather low affinity (especially high off-rate) towards the target protein (Table 3). In contrast mAb 21-5-4 strongly reduces the binding of HEK293 cells to ThrOpn. Furthermore, this mAb also inhibits binding of HEK cells to MmpOpn (FIG. 5B) again confirming its pan-reactivity against different Opn products. MMP cleavage site specific mAbs 7-5-4 and 9-3-1 inhibit exclusively MmpOpn induced adhesion of HEK cells (FIG. 5B) also in this case confirming data derived from ELISA and Western Blot analyses and thus proving their specificity.

### Example 5: Affinity Determination of mAbs 4-4-2, 7-5-4, 9-3-1, and 21-5-4 Using SPR

In order to define the binding strength of selected mAbs against their individual targets SPR analysis was performed using peptides representing the different Opn products. Monoclonal Ab 4-4-2 exhibited an affinity against ThrOpn of 13.9 nM and an off-rate value (describes the stability of the antibody-target-complex once formed) of 2,79 E-3 sec⁻¹. This off-rate value is rather high, indicating that the stability of the target-antibody complex is moderate. Thus in a situation where multiple washing steps are included (e.g. in the used functional cell based adhesion assay) the antibody will be washed away from its target. This is expected to be the reason why this antibody does not show inhibition of the HEK293 adhesion in the functional assay. On the other hand the mAb 4-4-2 shows medium to good affinity (KD 13.9 nM) and thus this antibody exhibits a high on-rate capacity, which represents the capacity and velocity to identify and bind its target. In a more physiological situation where antibody persistence is not influenced by steps depleting the antibody, mAb 4-4-2 is expected also display functional activity.

Monoclonal Abs 7-5-4 and 9-3-1 exhibit almost identical off-rate and KD values. In both cases the KD value is in the lowest nM or even high pM range, representing high affinity. An excellent off-rate value is exhibited by mAb 21-5-4. This antibody also displays the highest affinity against the target region.

TABLE 3
Affinity determination of mAbs against their targets

| Name of | | Off-rate | |
|---|---|---|---|
| clone | Opn product tested | values (sec⁻¹) | K_{d} values (nM) |
| 4-4-2 | ThrOpn | 2.79E−3 | 13.9E−9 |
| 7-5-4 | MmpOpn | 8.89E−5 | 1.12E−9 |
| 9-3-1 | MmpOpn | 8.79E−5 | 0.97E−9 |
| 21-5-4 | ThrOpn and MmpOpn | 1.00E−5 | 0.84E−9 |

Summarizing the mAb Results

Monoclonal Ab 4-4-2 specifically recognizes ThrOpn in its native (FIG. 3A) as well as in its denatured (FIG. 4A) form.

Monoclonal Abs 7-5-4 and 9-3-1 specifically recognizes MmpOpn in its native (FIGS. 3 B and C) as well as in its denatured (FIGS. 4B and C) form and are functionally active, leading to a clear inhibition of the MmpOpn-induced adhesion of HEK293 cells (FIG. 5B).

Monoclonal Ab 21-5-4 recognizes all Opn fragments in their native (FIG. 3D) as well as in their denatured (FIG. 4D) forms. Additionally, it is functionally active, as it leads to a clear inhibition of both, MmpOpn- and ThrOpn-induced adhesion of HEK293 cells.

### Example 6: Usage of the Inventive mAb for Diagnosis

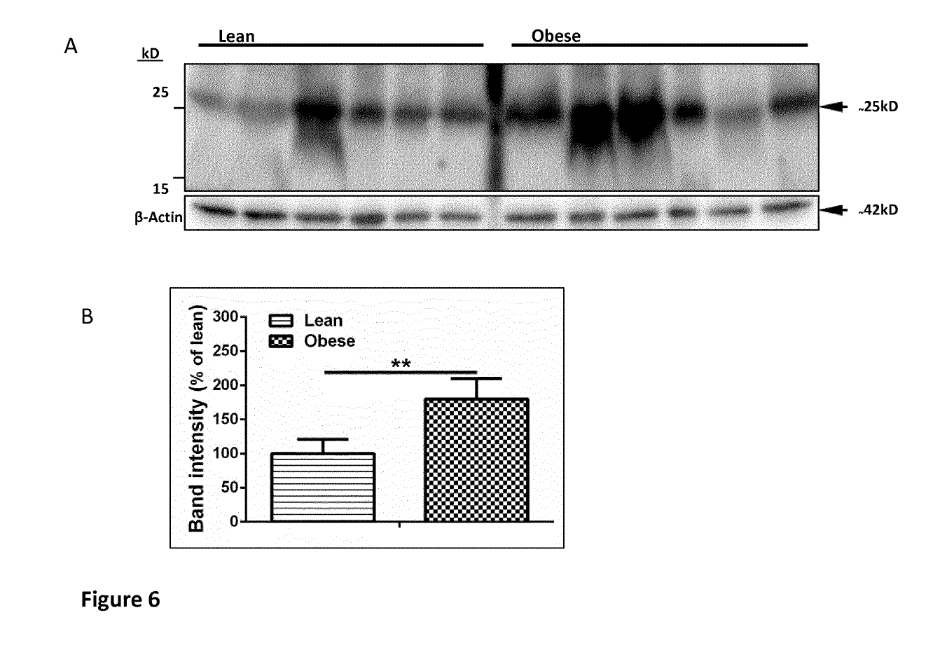

To test whether increased gene expression of Opn and MMPs is reflected in Opn protein and Opn fragment abundance in human adipose tissue (AT), we separated AT protein lysates from obese and controls by gel electrophoresis and probed for Opn using mAb 9-3-1. These mAb was shown to recognize predominantly Mmp-cleaved Opn. We found a significantly increased intensity of the 25 kD band corresponding to the C-terminal cleavage product normalized to beta actin expression (FIG. 6).

### Example 7: Monoclonal Antibody Sequencing

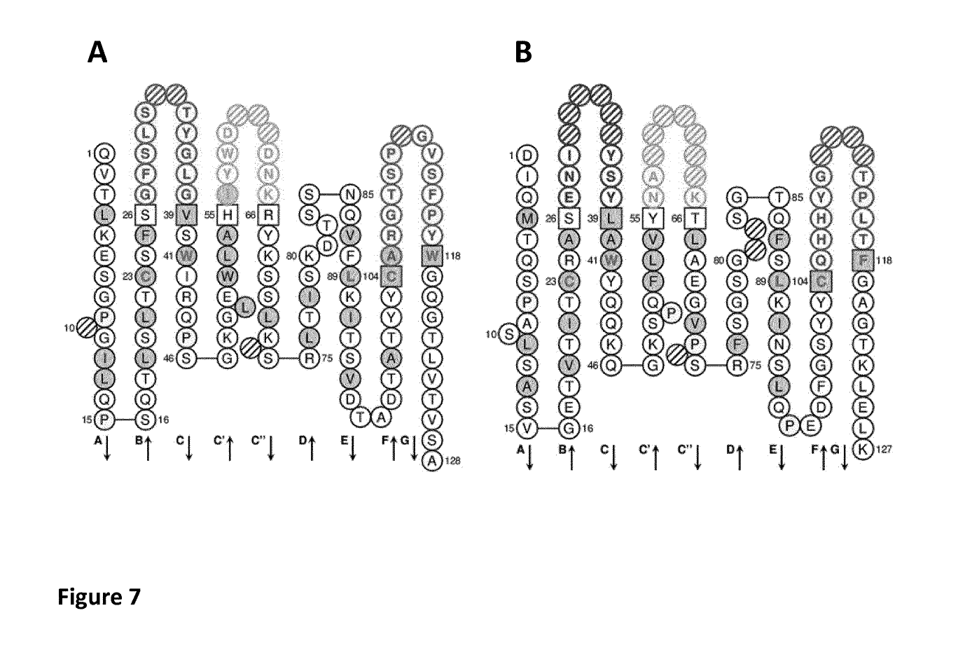

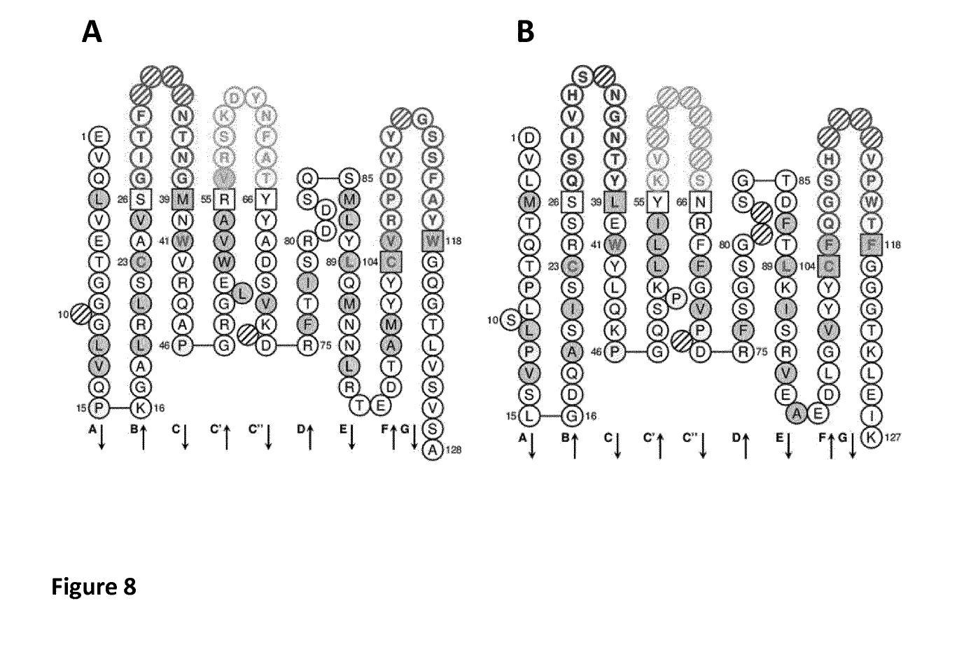

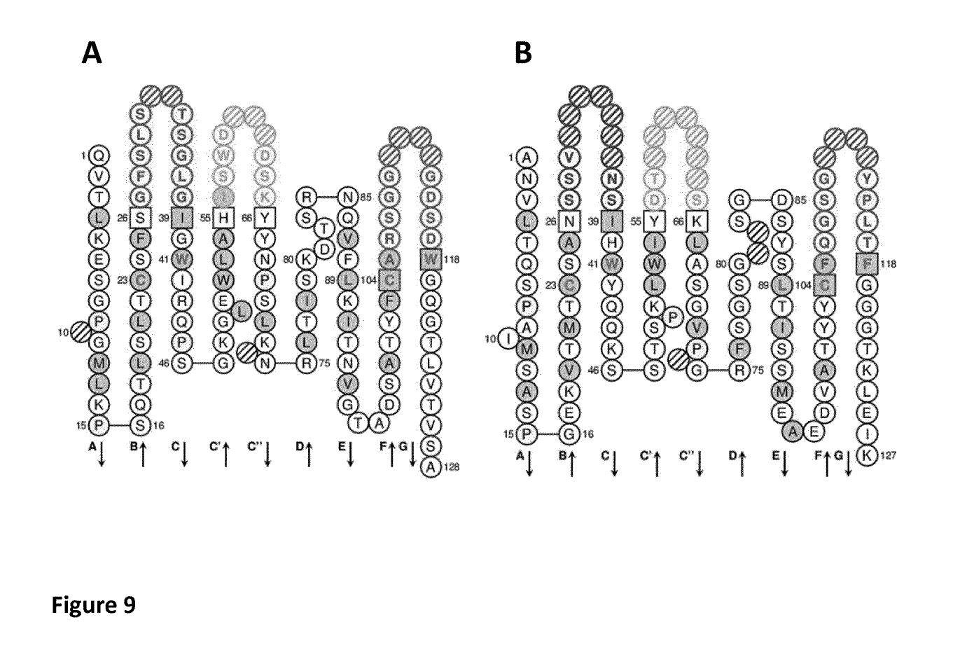

mRNA was extracted from the hybridoma cell pellets. Total RNA was extracted from the pellets. cDNA was created from the RNA by reverse-transcription with an oligo(dT) primer. The cDNA was S.N.A.P purified and the tailing of cDNA by TdT, were carried out using the 5′ RACE kit. PCR reactions were set up using AAP and variable domain reverse primers to amplify both the VH and VL regions of the monoclonal antibody DNA. The VH and VL products were cloned into the Invitrogen sequencing vector pCR2.1 and transformed into TOP10 cells and screened by PCR for positive transformants. Selected colonies were picked and analyzed by DNA sequencing on an ABI3130xl Genetic Analyzer, the result is shown in FIG. 7 (mAb 4-4-2), FIG. 8 (mAb 7-5-4 and 9-3-1, which turned out to be identical to mAb 7-5-4) and FIG. 9 (mAb 21-5-4).

The present invention is further exemplified by (but not limited to) the following embodiments:

1. A monoclonal antibody specific for one or more truncated variants of human osteopontin (Opn),
wherein the antibody is more reactive towards the one or more truncated variants than towards the full-length Opn (flOpn; SEQ ID NO: 15); and
wherein the antibody is specific for:
(A) matrix-metalloproteinase-truncated Opn (MmpOpn; SEQ ID NO: 16), wherein the antibody is more reactive towards MmpOpn (SEQ ID NO: 16) than towards each of flOpn (SEQ ID NO: 15) and thrombin-truncated Opn (ThrOpn; SEQ ID NO: 17); or
(B) both MmpOpn (SEQ ID NO: 16) and ThrOpn (SEQ ID NO: 17), wherein the antibody is more reactive towards each of MmpOpn (SEQ ID NO: 16) and ThrOpn (SEQ ID NO: 17) than towards flOpn (SEQ ID NO: 15); or
(C) ThrOpn (SEQ ID NO: 17), wherein the antibody is specific for an ThrOpn epitope with a peptide sequence selected from the group of VVYGLR, SVVYGLR and DSVVYGLR (SEQ ID NOs: 1-3), wherein, in case the antibody is specific for the epitope with the peptide sequence SVVYGLR, the antibody's variable domain of the heavy chain (V_{H}) and the antibody's variable domain of the light chain (V_{L}) comprise complementarity-determining regions (CDRs) with the following sequences:

V_{H} CDR1
(SEQ ID NO: 18)
GFSLSTYGLG,
V_{H} CDR2
(SEQ ID NO: 19)
IYWDDNK,
V_{H} CDR3
(SEQ ID NO: 20)
ARGTSPGVSFPY,
V_{L} CDR1
(SEQ ID NO: 21)
ENIYSY,
V_{L} CDR2
(SEQ ID NO: 22)
NAK,
V_{L} CDR3
(SEQ ID NO: 23)
QHHYGTPLT,

and wherein the antibody is more reactive towards ThrOpn (SEQ ID NO: 17) than towards each of flOpn (SEQ ID NO: 15) and MmpOpn (SEQ ID NO: 16) and preferably wherein V_{H} comprises the sequence of SEQ ID NO: 24 and V_{L} comprises the sequence of SEQ ID NO: 25.
2. The antibody of embodiment 1(A), wherein the antibody is specific for an MmpOpn epitope with a peptide sequence selected from the group of GDSVVYG, RGDSVVYG and DGRGDSVVYG (SEQ ID NOs: 7-9).
3. The antibody of embodiment 1(B), wherein the antibody is specific for a MmpOpn/ThrOpn epitope with a peptide sequence selected from the group of TYDGRGDSVVYG (SEQ ID NO: 10) and PTVDTYDGRGDS (SEQ ID NO: 14).
4. The antibody of embodiment 2, wherein the antibody is specific for the epitope with the peptide sequence GDSVVYG and the CDRs of the antibody comprise the following sequences:

V_{H} CDR1
(SEQ ID NO: 26)
GITFNTNG,
V_{H} CDR2
(SEQ ID NO: 27)
VRSKDYNFAT,
V_{H} CDR3
(SEQ ID NO: 28)
VRPDYYGSSFAY,
V_{L} CDR1
(SEQ ID NO: 29)
QSIVHSNGNTY,
V_{L} CDR2
(SEQ ID NO: 30)
KVS,
V_{L} CDR3
(SEQ ID NO: 31)
FQGSHVPWT,

and preferably wherein V_{H} comprises the sequence of SEQ ID NO: 32 and V_{L} comprises the sequence of SEQ ID NO: 33.
5. The antibody of embodiment 3, wherein the antibody is specific for the epitope with the peptide sequence TYDGRGDSVVYG and the CDRs of the antibody comprise the following sequences:

V_{H} CDR1
(SEQ ID NO: 34)
GFSLSTSGLG,
V_{H} CDR2
(SEQ ID NO: 35)
ISWDDSK,
V_{H} CDR3
(SEQ ID NO: 363)
ARSGGGDSD,
V_{L} CDR1
(SEQ ID NO: 37)
SSVNS,
V_{L} CDR2
(SEQ ID NO: 38)
DTS,
V_{L} CDR3
(SEQ ID NO: 39)
FQGSGYPLT

and preferably wherein V_{H} comprises the sequence of SEQ ID NO: 40 and V_{L} comprises the sequence of SEQ ID NO: 41.
6. The antibody of embodiment 1(C), 4 or 5, wherein three, preferably two, more preferably one, of the amino-acids of the CDR or V_{H} or V_{L} is mutated into any other amino-acid.
7. The antibody of any one of embodiments 1 to 6, wherein
in case of the antibody being specific for MmpOpn (SEQ ID NO: 16), the antibody is more than N times more reactive towards MmpOpn (SEQ ID NO: 16) than towards each of flOpn (SEQ ID NO: 15) and ThrOpn (SEQ ID NO: 17); and
in case of the antibody being specific for both MmpOpn (SEQ ID NO: 16) and ThrOpn (SEQ ID NO: 17), the antibody is more than N times more reactive towards each of MmpOpn (SEQ ID NO: 16) and ThrOpn (SEQ ID NO: 17) than towards flOpn (SEQ ID NO: 15); and
in case of the antibody being specific for ThrOpn (SEQ ID NO: 17), the
antibody is more than N times more reactive towards ThrOpn (SEQ ID NO: 17) than towards each of flOpn (SEQ ID NO: 15) and MmpOpn (SEQ ID NO: 16); and
   wherein N is more than 1.5, preferably more than 2, more preferably more than 3, even more preferably more than 5, most preferably more than 10; and
   preferably wherein the reactivity of the antibody towards MmpOpn (SEQ ID NO: 16), ThrOpn (SEQ ID NO: 17) and flOpn (SEQ ID NO: 15), respectively, is measured by ELISA on a plate coated by MmpOpn (SEQ ID NO: 16), ThrOpn (SEQ ID NO: 17) and flOpn (SEQ ID NO: 15), respectively, after blocking with 1% BSA comprising the following conditions:
concentration of the antibody: 0.25 μg/ml,
concentration of the secondary, HRP-coupled antibody: 0.1 μg/ml,
HRP substrate: ABTS and 0.1% hydrogen peroxide, read-out: absorbance at 405 nm.
   8. The antibody of any one of embodiments 1 to 7, wherein the dissociation constant K_{d} in regard to the respective epitope and/or the respective Opn protein is lower than 50 nM, preferably lower than 20 nM, more preferably lower than 10 nM, even more preferably lower than 5 nM, most preferably lower than 2 nM.
   9. The antibody of any one of embodiments 1 to 8, wherein the off-rate value in regard to the respective epitope and/or the respective Opn protein is lower than 5×10⁻³s⁻¹, preferably lower than 3×10⁻³s⁻¹, more preferably lower than 1×10⁻³s⁻¹, even more preferably lower than 1×10⁻⁴s⁻¹.
   10. The antibody of any one of embodiments 1 to 9, wherein the antibody is humanised.
   11. A fragment of the antibody of any one of embodiments 1 to 10, preferably a single-domain antibody, wherein the fragment is specific for:
   (A) MmpOpn (SEQ ID NO: 16), wherein the fragment is more reactive towards MmpOpn (SEQ ID NO: 16) than towards each of flOpn (SEQ ID NO: 15) and ThrOpn (SEQ ID NO: 17); or
   (B) both MmpOpn (SEQ ID NO: 16) and ThrOpn (SEQ ID NO: 17), wherein the fragment is more reactive towards each of MmpOpn (SEQ ID NO: 16) and ThrOpn (SEQ ID NO: 17) than towards flOpn (SEQ ID NO: 15); or
   (C) ThrOpn (SEQ ID NO: 17), wherein the fragment is more reactive towards ThrOpn (SEQ ID NO: 17) than towards each of flOpn (SEQ ID NO: 15) and MmpOpn (SEQ ID NO: 16).
   12. A pharmaceutical composition, comprising at least one antibody of any one of embodiments 1 to 10 and/or at least one fragment of embodiment 11 and at least one pharmaceutically acceptable excipient.
   13. A vaccine, comprising at least one isolated Opn peptide:
   (A) with one or more sequences selected from the group of GDSVVYG, RGDSVVYG and DGRGDSVVYG (SEQ ID NOs: 7-9) and GRGDSVVYG (SEQ ID NO: 55); and/or
   (B) with one or more sequences selected from the group of TYDGRGDSVVYG (SEQ ID NO: 10), VDTYDGRGDSVV (SEQ ID NO: 13), PTVDTYDGRGDS (SEQ ID NO: 14), DTYDGRGDSVVY (SEQ ID NO: 56) and TVDTYDGRGDSV (SEQ ID NO: 57), wherein the peptide, especially the peptide with the sequence VDTYDGRGDSVV (SEQ ID NO: 13) or PTVDTYDGRGDS (SEQ ID NO: 14), is preferably amidated at its C-terminus; and/or
   (C) with one or more sequences selected from the group of VVYGLR, SVVYGLR and DSVVYGLR (SEQ ID NOs: 1-3) and GDSVVYGLR (SEQ ID NO: 58);
   and wherein the peptides are coupled or fused to a pharmaceutically acceptable carrier, preferably a protein carrier and preferably wherein the peptides are covalently coupled to the carrier.
   14. The vaccine of embodiment 13, wherein the carrier is a protein, preferably selected from the group of keyhole limpet haemocyanin (KLH), tetanus toxoid (TT), protein D or diphtheria toxin (DT), especially KLH.
   15. The vaccine of embodiment 14, wherein the at least one peptide has an additional cystein residue at its N-terminus and/or C-terminus and the at least one peptide is covalently coupled to the protein carrier, or a linker coupled thereto, through the additional cystein residue, preferably wherein the linker contains a maleimide group or haloacetyl group that reacts with the cystein of the peptide.
   16. The vaccine of any one of embodiments 13 to 15, further comprising at least one pharmaceutically acceptable excipient and/or adjuvant.
   17. The composition of embodiment 12 or the vaccine of any one of embodiments 13 to 16 for use in therapy.
   18. The composition of embodiment 12 or the vaccine of any one of embodiments 13 to 16 for use in treatment and/or prevention of cardiovascular disease, in particular of atherosclerosis.
   19. The composition of embodiment 12 or the vaccine of any one of embodiments 13 to 16 for use in treatment and/or prevention of Type-2 diabetes, in particular of obesity-related insulin resistance.
   20. A method for manufacturing the antibody of any one of embodiments 1 to 10, comprising:
expressing the antibody in cell culture; and
purifying the antibody.
   21. A method for manufacturing the vaccine of any one of embodiments 13 to 16, comprising:
providing the peptides; and
coupling the peptides to the carrier, preferably KLH protein; and
optionally, adding pharmaceutically acceptable excipients.
   22. A diagnostic method, comprising:
providing an isolated sample of the patient, preferably a sample from blood and/or adipose tissue, especially from subcutaneous adipose tissue; and
using the antibody of any one of embodiments 1 to 10 to measure levels of ThrOpn, MmpOpn and/or the aggregate level of ThrpOpn and MmpOpn in the sample, preferably by an ELISA or Western blot; and
comparing these levels to levels of a healthy control population and/or to levels of the patient at an earlier time point; and
create a diagnosis or prognosis of a disease or condition or of the progression of said disease or condition, preferably cardiovascular disease, in particular atherosclerosis, or type-2 diabetes, in particular obesity-related insulin resistance.
   23. Use of the method of embodiment 22 to monitor efficacy of a therapy according to any one of embodiments 17 to 19.

1. A monoclonal antibody specific for one or more truncated variants of human osteopontin (Opn),
wherein the antibody is more reactive towards the one or more truncated variants than towards the full-length Opn (flOpn; SEQ ID NO: 15); and
wherein the antibody is specific for:
(A) matrix-metalloproteinase-truncated Opn (MmpOpn; SEQ ID NO: 16), wherein the antibody is more reactive towards MmpOpn (SEQ ID NO: 16) than towards each of flOpn (SEQ ID NO: 15) and thrombin-truncated Opn (ThrOpn; SEQ ID NO: 17); or
(B) both MmpOpn (SEQ ID NO: 16) and ThrOpn (SEQ ID NO: 17), wherein the antibody is more reactive towards each of MmpOpn (SEQ ID NO: 16) and ThrOpn (SEQ ID NO: 17) than towards flOpn (SEQ ID NO: 15); or
(C) ThrOpn (SEQ ID NO: 17), wherein the antibody is specific for an ThrOpn epitope with a peptide sequence selected from the group consisting of VVYGLR (SEQ ID NO: 1), SVVYGLR (SEQ ID NO: 2) and DSVVYGLR (SEQ ID NO: 3), wherein, in case the antibody is specific for the epitope with the peptide sequence SVVYGLR (SEQ ID NO: 2), the antibody's variable domain of the heavy chain (V_{H}) and the antibody's variable domain of the light chain (V_{L}) comprise complementarity-determining regions (CDRs) with the following sequences:
V_{H} CDR1
(SEQ ID NO: 18)
GFSLSTYGLG,
V_{H} CDR2
(SEQ ID NO: 19)
IYWDDNK,
V_{H} CDR3
(SEQ ID NO: 20)
ARGTSPGVSFPY,
V_{L} CDR1
(SEQ ID NO: 21)
ENIYSY,
V_{L} CDR2
(SEQ ID NO: 22)
NAK,
V_{L} CDR3
(SEQ ID NO: 23)
QHHYGTPLT,
and
wherein the antibody is more reactive towards ThrOpn (SEQ ID NO: 17) than towards each of flOpn (SEQ ID NO: 15) and MmpOpn (SEQ ID NO: 16) and, optionally, wherein V_{H} comprises the sequence of SEQ ID NO: 24 and V_{L} comprises the sequence of SEQ ID NO: 25.

2. The antibody of claim 1, wherein (A) the antibody is specific for an MmpOpn epitope with a peptide sequence selected from the group consisting of GDSVVYG (SEQ ID NO: 7), RGDSVVYG (SEQ ID NO: 8) and DGRGDSVVYG (SEQ ID NO: 9).

3. The antibody of claim 1, wherein (B) the antibody is specific for a MmpOpn/ThrOpn epitope with a peptide sequence selected from the group consisting of TYDGRGDSVVYG (SEQ ID NO: 10) and PTVDTYDGRGDS (SEQ ID NO: 14).

4. The antibody of claim 2, wherein the antibody is specific for the epitope with the peptide sequence GDSVVYG (SEQ ID NO: 7) and the CDRs of the antibody comprise the following sequences:
V_{H} CDR1
(SEQ ID NO: 26)
GITFNTNG,
V_{H} CDR2
(SEQ ID NO: 27)
VRSKDYNFAT,
V_{H} CDR3
(SEQ ID NO: 28)
VRPDYYGSSFAY,
V_{L} CDR1
(SEQ ID NO: 29)
QSIVHSNGNTY,
V_{L} CDR2
(SEQ ID NO: 30)
KVS,
V_{L} CDR3
(SEQ ID NO: 31)
FQGSHVPWT,
and,
optionally, wherein V_{H} comprises the sequence of SEQ ID NO: 32 and V_{L} comprises the sequence of SEQ ID NO: 33.

5. The antibody of claim 3, wherein the antibody is specific for the epitope with the peptide sequence TYDGRGDSVVYG (SEQ ID NO: 10) and the CDRs of the antibody comprise the following sequences:
V_{H} CDR1
(SEQ ID NO: 34)
GFSLSTSGLG,
V_{H} CDR2
(SEQ ID NO: 35)
ISWDDSK,
V_{H} CDR3
(SEQ ID NO: 363)
ARSGGGDSD,
V_{L} CDR1
(SEQ ID NO: 37)
SSVNS,
V_{L} CDR2
(SEQ ID NO: 38)
DTS,
V_{L} CDR3
(SEQ ID NO: 39)
FQGSGYPLT
and, optionally, wherein V_{H} comprises the sequence of SEQ ID NO: 40 and V_{L} comprises the sequence of SEQ ID NO: 41.

6. The antibody of claim 1, wherein one, two or three of the amino-acids of the CDR or V_{H} or V_{L} is mutated into any other amino-acid.

7. The antibody of claim 1, wherein
in case of the antibody being specific for MmpOpn (SEQ ID NO: 16), the antibody is more than N times more reactive towards MmpOpn (SEQ ID NO: 16) than towards each of flOpn (SEQ ID NO: 15) and ThrOpn (SEQ ID NO: 17); and
in case of the antibody being specific for both MmpOpn (SEQ ID NO: 16) and ThrOpn (SEQ ID NO: 17), the antibody is more than N times more reactive towards each of MmpOpn (SEQ ID NO: 16) and ThrOpn (SEQ ID NO: 17) than towards flOpn (SEQ ID NO: 15); and
in case of the antibody being specific for ThrOpn (SEQ ID NO: 17), the antibody is more than N times more reactive towards ThrOpn (SEQ ID NO: 17) than towards each of flOpn (SEQ ID NO: 15) and MmpOpn (SEQ ID NO: 16); and
wherein N is more than 1.5.

8. The antibody of claim 1,
wherein the dissociation constant K_{d} in regard to the respective epitope and/or in regard to the respective Opn protein is lower than 50 nM; and/or
wherein the off-rate value in regard to the respective epitope and/or in regard to the respective Opn protein is lower than 5×10⁻³s⁻¹; and/or
wherein the antibody is humanised.

9. A fragment of the antibody of claim 1, wherein the fragment is specific for:
(A) MmpOpn (SEQ ID NO: 16), wherein the fragment is more reactive towards MmpOpn (SEQ ID NO: 16) than towards each of flOpn (SEQ ID NO: 15) and ThrOpn (SEQ ID NO: 17); or
(B) both MmpOpn (SEQ ID NO: 16) and ThrOpn (SEQ ID NO: 17), wherein the fragment is more reactive towards each of MmpOpn (SEQ ID NO: 16) and ThrOpn (SEQ ID NO: 17) than towards flOpn (SEQ ID NO: 15); or
(C) ThrOpn (SEQ ID NO: 17), wherein the fragment is more reactive towards ThrOpn (SEQ ID NO: 17) than towards each of flOpn (SEQ ID NO: 15) and MmpOpn (SEQ ID NO: 16).

10. A pharmaceutical composition comprising at least one antibody of claim 1 or a human osteopontin (Opn)-binding fragment thereof and at least one pharmaceutically acceptable excipient.

11. A vaccine comprising at least one isolated Opn peptide:
(A) with one or more sequences selected from the group consisting of GDSVVYG (SEQ ID NO: 7), RGDSVVYG (SEQ ID NO: 8) and DGRGDSVVYG (SEQ ID NO: 9) and GRGDSVVYG (SEQ ID NO: 55); and/or
(B) with one or more sequences selected from the group consisting of TYDGRGDSVVYG (SEQ ID NO: 10), VDTYDGRGDSVV (SEQ ID NO: 13), PTVDTYDGRGDS (SEQ ID NO: 14), DTYDGRGDSVVY (SEQ ID NO: 56) and TVDTYDGRGDSV (SEQ ID NO: 57); and/or
(C) with one or more sequences selected from the group consisting of VVYGLR (SEQ ID NO: 1), SVVYGLR (SEQ ID NO: 2) and DSVVYGLR (SEQ ID NO: 3) and GDSVVYGLR (SEQ ID NO: 58); and
wherein the peptides are coupled or fused to a pharmaceutically acceptable.

12. The vaccine of claim 11, wherein the pharmaceutically acceptable carrier is limpet haemocyanin (KLH), tetanus toxoid (TT), protein D, diphtheria toxin (DT) or another protein carrier.

13. A method for manufacturing the antibody of claim 1, comprising:
expressing the antibody in cell culture; and
purifying the antibody.

14. A method for manufacturing the vaccine of claim 11, comprising:
providing the at least one peptide; and
coupling the at least one peptide to the carrier; and
optionally, adding one or more pharmaceutically acceptable excipients.

15. A diagnostic method, comprising:
providing an isolated sample of the patient; and
using the antibody of claim 1 or a human osteopontin (Opn)-binding fragment thereof to measure levels of ThrOpn, MmpOpn and/or the aggregate level of ThrpOpn and MmpOpn in the sample; and
comparing these levels to levels of a healthy control population and/or to levels of the patient at an earlier time point; and
creating a diagnosis or prognosis of a disease or condition or of the progression of said disease or condition.

16. A method for preventing or treating a disease, disorder or condition associated with an osteopontin comprising administering the antibody of claim 1, or a human osteopontin (Opn)-binding fragment thereof to a subject in need thereof.

17. The method of claim 16, wherein said disease, disorder or condition is atherosclerosis or another cardiovascular disease; or is obesity-related insulin resistance or Type-2 diabetes.

18. A method for preventing or treating a disease, disorder or condition associated with an osteopontin comprising administering the vaccine of claim 11 to a subject in need thereof.

19. The method of claim 18, wherein said disease, disorder or condition is atherosclerosis or another cardiovascular disease; or is obesity-related insulin resistance or Type-2 diabetes.


SEQUENCE LISTING (58 sequences)

SEQ ID NO: 1 - protein, 6 aa, Artificial Sequence
  Peptide epitope
VVYGLR

SEQ ID NO: 2 - protein, 7 aa, Artificial Sequence
  Peptide epitope
SVVYGLR

SEQ ID NO: 3 - protein, 8 aa, Artificial Sequence
  Peptide epitope
DSVVYGLR

SEQ ID NO: 4 - protein, 10 aa, Artificial Sequence
  Peptide epitope
RGDSVVYGLR

SEQ ID NO: 5 - protein, 12 aa, Artificial Sequence
  Peptide epitope
DGRGDSVVYGLR

SEQ ID NO: 6 - protein, 6 aa, Artificial Sequence
  Peptide epitope
DSVVYG

SEQ ID NO: 7 - protein, 7 aa, Artificial Sequence
  Peptide epitope
GDSVVYG

SEQ ID NO: 8 - protein, 8 aa, Artificial Sequence
  Peptide epitope
RGDSVVYG

SEQ ID NO: 9 - protein, 10 aa, Artificial Sequence
  Peptide epitope
DGRGDSVVYG

SEQ ID NO: 10 - protein, 12 aa, Artificial Sequence
  Peptide epitope
TYDGRGDSVVYG

SEQ ID NO: 11 - protein, 12 aa, Artificial Sequence
  Peptide epitope
DGRGDSVVYGLR

SEQ ID NO: 12 - protein, 12 aa, Artificial Sequence
  Peptide epitope
TYDGRGDSVVYG

SEQ ID NO: 13 - protein, 12 aa, Artificial Sequence
  Peptide epitope
VDTYDGRGDSVV

SEQ ID NO: 14 - protein, 12 aa, Artificial Sequence
  Peptide epitope
PTVDTYDGRGDS

SEQ ID NO: 15 - protein, 314 aa, Homo sapiens
  Osteopontin (Opn)
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 16 - protein, 166 aa, Homo sapiens
  MmpOpn
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYG

SEQ ID NO: 17 - protein, 168 aa, Homo sapiens
  ThrOpn
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLR

SEQ ID NO: 18 - protein, 10 aa, Artificial Sequence
  mAb 4-4-2 VH CDR1
GFSLSTYGLG

SEQ ID NO: 19 - protein, 7 aa, Artificial Sequence
  mAb 4-4-2 VH CDR2
IYWDDNK

SEQ ID NO: 20 - protein, 12 aa, Artificial Sequence
  mAb 4-4-2 VH CDR3
ARGTSPGVSFPY

SEQ ID NO: 21 - protein, 6 aa, Artificial Sequence
  mAb 4-4-2 VL CDR1
ENIYSY

SEQ ID NO: 22 - protein, 3 aa, Artificial Sequence
  mAb 4-4-2 VL CDR2
NAK

SEQ ID NO: 23 - protein, 9 aa, Artificial Sequence
  mAb 4-4-2 VL CDR3
QHHYGTPLT

SEQ ID NO: 24 - protein, 151 aa, Artificial Sequence
  mAb 4-4-2 VH
MNRLTSSLLLLIVPAYVLSQVTLKESGPGILQPSQTLSLTCSFSGFSLSTYGLGVSWIRQPSGKGLEWLAHIYWDDNKRYKSSLKSRLTISKDTSSNQVFLKITSVDTADTATYYCARGTSPGVSFPYWGQGTLVTVSAAKTTPPSVYPLA

SEQ ID NO: 25 - protein, 162 aa, Artificial Sequence
  mAb 4-4-2 VL
MSVPTQVLGLLLLWLTGARCDIQMTQSPASLSASVGETVTITCRASENIYSYLAWYQQKQGKSPQFLVYNAKTLAEGVPSRFSGSGSGTQFSLKINSLQPEDFGSYYCQHHYGTPLTFGAGTKLELKRADAAPTVSIFPPSSEQLTSGGASVVCFLNNFYPK

SEQ ID NO: 26 - protein, 8 aa, Artificial Sequence
  mAb 7-5-4 VH CDR1
GITFNTNG

SEQ ID NO: 27 - protein, 10 aa, Artificial Sequence
  mAb 7-5-4 VH CDR2
VRSKDYNFAT

SEQ ID NO: 28 - protein, 12 aa, Artificial Sequence
  mAb 7-5-4 VH CDR3
VRPDYYGSSFAY

SEQ ID NO: 29 - protein, 11 aa, Artificial Sequence
  mAb 7-5-4 VL CDR1
QSIVHSNGNTY

SEQ ID NO: 30 - protein, 3 aa, Artificial Sequence
  mAb 7-5-4 VL CDR2
KVS

SEQ ID NO: 31 - protein, 9 aa, Artificial Sequence
  mAb 7-5-4 VL CDR3
FQGSHVPWT

SEQ ID NO: 32 - protein, 153 aa, Artificial Sequence
  mAb 7-5-4 VH
MLLGLKWVFFVVFYQGVHCEVQLVETGGGLVQPKGALRLSCAVSGITFNTNGMNWVRQAPGRGLEWVARVRSKDYNFATYYADSVKDRFTISRDDSQSMLYLQMNNLRTEDTAMYYCVRPDYYGSSFAYWGQGTLVSVSAAKTTPPPVYPLAP

SEQ ID NO: 33 - protein, 166 aa, Artificial Sequence
  mAb 7-5-4 VL
MKLPVRLLVLMFWIPASSSDVLMTQTPLSLPVSLGDQASISCRSSQSIVHSNGNTYLEWYLQKPGQSPKLLIYKVSNRFFGVPDRFSGSGSGTDFTLKISRVEAEDLGVYYCFQGSHVPWTFGGGTKLEIKRADAAPTVSIFPPSSEQLTSGGASVVCFLNNFYPR

SEQ ID NO: 34 - protein, 10 aa, Artificial Sequence
  mAb 21-5-4 VH CDR1
GFSLSTSGLG

SEQ ID NO: 35 - protein, 7 aa, Artificial Sequence
  mAb 21-5-4 VH CDR2
ISWDDSK

SEQ ID NO: 36 - protein, 9 aa, Artificial Sequence
  mAb 21-5-4 VH CDR3
ARSGGGDSD

SEQ ID NO: 37 - protein, 5 aa, Artificial Sequence
  mAb 21-5-4 VL CDR1
SSVNS

SEQ ID NO: 38 - protein, 3 aa, Artificial Sequence
  mAb 21-5-4 VL CDR2
DTS

SEQ ID NO: 39 - protein, 9 aa, Artificial Sequence
  mAb 21-5-4 VL CDR3
FQGSGYPLT

SEQ ID NO: 40 - protein, 149 aa, Artificial Sequence
  mAb 21-5-4 VH
MDRLTSSFLLLIVPAYVLSQVTLKESGPGMLKPSQTLSLTCSFSGFSLSTSGLGIGWIRQPSGKGLEWLAHISWDDSKYYNPSLKNRLTISKDTSRNQVFLKITNVGTADSATYFCARSGGGDSDWGQGTLVTVSAAKTTPPSVFPLAP

SEQ ID NO: 41 - protein, 163 aa, Artificial Sequence
  mAb 21-5-4 VL
MDFQVQIFSFLLISASVIMSRGANVLTQSPAIMSASPGEKVTMTCSANSSVNSIHWYQQKSSTSPKLWIYDTSKLASGVPGRFSGSGSGDSYSLTISSMEAEDVATYYCFQGSGYPLTFGGGTKLEIKRADAAPTVSIFPPSSEQLTSGGASVVCFLNNFYPK

SEQ ID NO: 42 - DNA, 38 bp, Artificial Sequence
  Primer
agcggctcttcaatgataccagttaaacaggctgattc

SEQ ID NO: 43 - DNA, 36 bp, Artificial Sequence
  Primer
agcggctcttctcccattgacctcagaagatgcact

SEQ ID NO: 44 - DNA, 39 bp, Artificial Sequence
  Primer
agcggctcttctcccctatccataaaccacactatcacc

SEQ ID NO: 45 - DNA, 38 bp, Artificial Sequence
  Primer
agcggctcttctcccctacctcagtccataaaccacac

SEQ ID NO: 46 - protein, 7 aa, Artificial Sequence
  Mouse-derived peptide epitope
SLAYGLR

SEQ ID NO: 47 - protein, 16 aa, Artificial Sequence
  Mouse-derived peptide epitope
VDVPNGRGDSLAYGLR

SEQ ID NO: 48 - protein, 17 aa, Artificial Sequence
  Peptide epitope
VDTYDGRGDSVVYGLRS

SEQ ID NO: 49 - protein, 8 aa, Artificial Sequence
  Peptide epitope
CSVVYGLR

SEQ ID NO: 50 - protein, 4 aa, Artificial Sequence
  Peptipe epitope
YGLR

SEQ ID NO: 51 - protein, 7 aa, Artificial Sequence
  Peptide epitope
VYGLRSK

SEQ ID NO: 52 - protein, 7 aa, Artificial Sequence
  Peptide epitope
YDGRGDS

SEQ ID NO: 53 - protein, 12 aa, Artificial Sequence
  Peptide epitope
TYDGAAASVVYG

SEQ ID NO: 54 - protein, 12 aa, Artificial Sequence
  Peptide epitope
TAAARGDAAAYG

SEQ ID NO: 55 - protein, 9 aa, Artificial Sequence
  Peptide epitope
GRGDSVVYG

SEQ ID NO: 56 - protein, 12 aa, Artificial Sequence
  Peptide epitope
DTYDGRGDSVVY

SEQ ID NO: 57 - protein, 12 aa, Artificial Sequence
  Peptide epitope
TVDTYDGRGDSV

SEQ ID NO: 58 - protein, 9 aa, Artificial Sequence
  Peptide epitope
GDSVVYGLR